(19) United States
(12) Patent Application Publication
(10) Pub. No.: US 2021/0052875 A1
(43) Pub. Date: Feb. 25, 2021

(54) BIODIFFUSION CHAMBER

(71) Applicants: Brendan LAURENZI, Rutland, MA (US); THOMAS JEFFERSON UNIVERSITY, Philadephia, PA (US); IMVAX, INC., Philadelphia, PA (US)

(72) Inventors: Brendan LAURENZI, Rutland, MA (US); David ANDREWS, Philadelphia, PA (US); William HITCHINGS, Wyndmoor, PA (US); Dominic VENTURA, Wilmington, NC (US); Kara PIGOTT, Philadelphia, PA (US); Douglas Craig Hooper, Medford, NJ (US)

(21) Appl. No.: 16/964,693

(22) PCT Filed: Jan. 24, 2019
(86) PCT No.: PCT/US2019/014961

§ 371 Date: Jul. 24, 2020

Related U.S. Application Data
(60) Provisional application No. 62/621,295, filed on Jan. 24, 2018.

(51) Int. Cl. A61M 39/02; C12N 15/113; A61K 35/13; A61K 31/7125; A61K 9/00

(52) U.S. Cl. CPC A61M 39/0208; C12N 15/1138; A61K 35/13; A61K 31/7125; A61K 9/0019; C12N 2320/31; A61M 2206/00; C12N 2320/35; C12N 2310/11; C12N 2310/315; C12N 2320/32; A61M 2039/0223

(57) ABSTRACT

A biodiffusion chamber for performing autologous cell vaccination is provided. The biodiffusion chamber is adapted for insertion into and removal from a subject. In some embodiments, the biodiffusion chamber comprises (i) a chamber body defining a hollow cavity and including a first surface and a second surface, (ii) a first semi-permeable membrane coupled to the first surface, (iii) a second semi-permeable membrane coupled to the second surface, and (iv) an element and/or feature adapted for removing the biodiffusion chamber from the subject. The first semi-permeable membrane and the second semi-permeable membrane are permeable to fluids and soluble factors but are not permeable to cells. In some embodiments, composition comprising a therapeutically effective amount of an antisense oligodeoxynucleotide is inserted into the biodiffusion chamber and allowed to diffuse out of the biodiffusion chamber and into the subject via at least one of the first semi-permeable membrane or the second semi-permeable membrane.

### CROSS-REFERENCE TO RELATED APPLICATIONS

This application claims priority to and the benefit of U.S. Provisional Patent Application Ser. No. 62/621,295, filed Jan. 24, 2018, entitled, “Biodiffusion Chamber,” the disclosure of which is incorporated herein by reference in its entirety.

### FIELD OF THE DISCLOSURE

The present disclosure relates to the fields of medical devices and medicine. More particularly, the disclosure relates to a biodiffusion chamber adapted for insertion into and removal from a subject.

### SEQUENCE LISTING

The instant application contains a Sequence Listing, which has been submitted electronically in ASCII format and is hereby incorporated by reference in its entirety. Said ASCII copy, created on Jan. 23, 2019, is named IMVX_006_01WO_SeqList_ST25.txt and is 6 kilobytes in size.

### BACKGROUND

Implantable biodiffusion chambers are used for various applications including systemic and local drug delivery, gene therapy, and autologous cell vaccination. Biodiffusion chambers may be implanted into a subject during surgery, and removed after a therapeutically effective amount of time.

Biodiffusion chambers known in the art are often difficult to remove, increasing the chances of unnecessary harm to the patient during removal. For example, biodiffusion chambers known in the art must be grasped by their full diameter with forceps during removal, increasing the risk of misdirecting the forceps and puncturing the chamber. Biodiffusion chambers that are located deep within the patient may require digital removal, causing discomfort to the patient. Accordingly, there remains a need in the art for an improved biodiffusion chamber constructed to facilitate removal of the chamber from the subject.

### SUMMARY

In some embodiments, a biodiffusion chamber configured for insertion into and removal from a body includes a chamber body, a first semi-permeable membrane, and a second semi-permeable membrane. The chamber body includes a first surface and a second surface. The chamber body defines a hollow cavity configured to at least temporarily contain an amount of a composition, for example a composition including at least a mixture of cells and antisense molecules. A portion of the chamber body is configured to be engaged by a removal member configured to enable removal of the biodiffusion chamber from the body. The first semi-permeable membrane is configured to be coupled to the first surface of the chamber body and the second semi-permeable membrane is configured to be coupled to the second surface of the chamber body. In some embodiments, each of the first semi-permeable membrane and the second semi-permeable membrane is permeable to antisense molecules and impermeable to cells.

In some embodiments, a biodiffusion chamber configured for insertion into and removal from a subject, the biodiffusion chamber comprises a chamber body including a first surface, a second surface, and a flange, the flange defining an opening configured to receive at least a portion of a removal member. The chamber body defines a hollow cavity and an injection port in fluid communication with the hollow cavity, wherein the injection port is configured to convey a composition, for example a composition including at least an amount of a biologic factor, into the hollow cavity. The biodiffusion chamber also comprises a first semi-permeable membrane in contact with the first surface, a second semi-permeable membrane in contact with the second surface, a first retainer fixedly coupled to the first surface such that a portion of the first semi-permeable membrane is disposed between the first retainer and the first surface, and a second retainer fixedly coupled to the second surface such that a portion of the second semi-permeable membrane is disposed between the second retainer and the second surface. In some embodiments, the first semi-permeable membrane and the second semi-permeable membrane are permeable to the biologic factor and impermeable to cells.

In some embodiments, a method of manufacturing a biodiffusion chamber comprises (i) forming a chamber body, the chamber body defining a hollow cavity and an injection port in fluid communication with the hollow cavity, the chamber body having a first surface, a second surface, and a flange, the flange defining an opening, (ii) placing a first semi-permeable membrane in contact with the first surface of the chamber body, (iii) placing a second semi-permeable membrane in contact with the second surface of the chamber body (iv) coupling a first retainer to the first surface of the chamber body such that a portion of the first semi-permeable membrane is fixedly disposed between the first surface and the first retainer, (v) coupling a second retainer to the second surface of the chamber body such that a portion of the second semi-permeable membrane is fixedly disposed between the second surface and the second retainer, (vi) conveying, via the injection port, a composition (e.g., a composition including a mixture of cells and an antisense molecule) into the hollow cavity after the coupling of the first retainer to the first surface and the coupling of the second retainer to the second surface; and (vii) sealing the injection port after the conveying.

In some embodiments, a method of treating a patient in need thereof comprises administering to the patient a biodiffusion chamber of the disclosure. In some embodiments, the patient suffers from cancer, such as glioma or other solid tumor.

### BRIEF DESCRIPTION OF THE DRAWINGS

The following drawings form part of the present specification and are included to further demonstrate certain aspects of the present disclosure. The disclosure may be better understood by reference to these drawings in combination with the detailed description of specific embodiments presented herein.

FIG. 1 is a perspective schematic illustration of a biodiffusion chamber adapted for insertion into and removal from a subject, according to an embodiment.

FIG. 2 is a perspective schematic illustration of a biodiffusion chamber adapted for insertion into and removal from a subject, according to an embodiment.

FIG. 3 is a perspective illustration of a biodiffusion chamber adapted for insertion into and removal from a subject, according to an embodiment.

FIG. 4 is an exploded perspective view of the biodiffusion chamber of FIG. 3.

FIGS. 5-7 are a top view, a rear perspective view, and a side view of a chamber body included in the biodiffusion chamber of FIG. 3.

FIG. 8 is a cross-sectional view of the chamber body of FIGS. 5-7 illustrating an injection port.

FIGS. 9 and 10 are perspective views a first retainer and a second retainer included in the biodiffusion chamber of FIG. 3.

FIG. 11 is a cross-sectional view of the biodiffusion chamber of FIG. 3.

FIGS. 12 and 13 are a side perspective view and a rear view of a plug configured for use with the biodiffusion chamber of FIG. 3.

FIG. 14 is a cross-sectional view of the plug shown in FIG. 12.

FIG. 15 is a cross-sectional view of the plug of FIG. 12 inserted into the injection port of the chamber body shown in FIG. 8.

FIG. 16 is a flowchart illustrating a method of manufacturing a biodiffusion chamber according to an embodiment.

### DETAILED DESCRIPTION

In some embodiments, a biodiffusion chamber includes (i) a chamber body defining a hollow cavity and having a first surface, and a second surface; (ii) a first semi-permeable membrane coupled to the first surface; (iii) a second semi-permeable membrane coupled to the second surface; and (iii) an element and/or feature adapted for removing and/or facilitating a process of removing the biodiffusion chamber from the human body. In some embodiments, the element and/or feature is a hole in the chamber body (or a surface of the chamber body that defines the hole) that extends between the first surface to the second surface of the chamber. The biodiffusion chambers of the disclosure may be used for various applications including but not limited to systemic and local drug delivery, gene therapy, autologous cell vaccination, and/or the like.

Other objects, features, and/or advantages of the present disclosure will become apparent from the following detailed description. It should be understood, however, that the detailed description and the specific examples, while indicating specific embodiments of the disclosure, are given by way of illustration only. Various changes and/or modifications within the spirit and scope of the disclosure will become apparent to those skilled in the art from this detailed description.

In some embodiments, a biodiffusion chamber configured for insertion into and removal from a body includes a chamber body, a first semi-permeable membrane, and a second semi-permeable membrane. The chamber body includes a first surface and a second surface. The chamber body defines a hollow cavity configured to at least temporarily contain an amount of a composition (e.g., a composition including at least a mixture of cells and antisense molecules). A portion of the chamber body is configured to be engaged by a removal member configured to enable removal of the biodiffusion chamber from the body. The first semi-permeable membrane is configured to be coupled to the first surface of the chamber body and the second semi-permeable membrane is configured to be coupled to the second surface of the chamber body. Each of the first semi-permeable membrane and the second semi-permeable membrane is permeable to the antisense molecules and impermeable to the cells.

In some embodiments, a biodiffusion chamber configured for insertion into and removal from a body includes a chamber body, a first semi-permeable membrane, a second semi-permeable membrane, a first retainer, and a second retainer. The chamber body includes a first surface, a second surface, and a flange. The flange defines an opening configured to receive at least a portion of a removal member. The chamber body defines a hollow cavity and an injection port in fluid communication with the hollow cavity and configured to convey an amount of a composition, such as a composition including at least a biologic factor into the hollow cavity. The first semi-permeable membrane is in contact with the first surface and the first retainer is fixedly coupled to the first surface such that a portion of the first semi-permeable membrane is disposed between the first retainer and the first surface. The second semi-permeable membrane is in contact with the second surface and the second retainer is fixedly coupled to the second surface such that a portion of the second semi-permeable membrane is disposed between the second retainer and the second surface. Each of the first semi-permeable membrane and the second semi-permeable membrane is permeable to at least a portion of the composition and impermeable to cells.

In some embodiments, a method of manufacturing a biodiffusion chamber includes forming a chamber body such that the chamber body (i) defines a hollow cavity and an injection port in fluid communication with the hollow cavity and (ii) has a first surface, a second surface, and a flange that defines an opening. A first semi-permeable membrane is placed in contact with the first surface of the chamber body and a second semi-permeable membrane is placed in contact with the second surface of the chamber body. A first retainer is coupled to the first surface of the chamber body such that a portion of the first semi-permeable membrane is fixedly disposed between the first surface and the first retainer. A second retainer is coupled to the second surface of the chamber body such that a portion of the second semi-permeable membrane is fixedly disposed between the second surface and the second retainer. An amount of a composition (e.g., a composition including at least a mixture of cells and antisense molecules) is conveyed into the hollow cavity via the injection port and after the first retainer is coupled to the first surface and the second retainer is coupled to the second surface. The injection port is sealed after conveying the composition.

As used in this specification, the singular forms “a,” “an,” and “the” include plural referents unless the context clearly dictates otherwise. Thus, for example, the term “a member” is intended to mean a single member or a combination of members, “a material” is intended to mean one or more materials, or a combination thereof.

All of the ranges listed herein are intended to include the endpoint values. For example, a range of whole numbers between 5 and 10 includes the values 5, 6, 7, 8, 9, and 10. The terms “about” and “approximately” refer to a range of plus or minus 10% of the indicated value. For example, about 0.5 would include 0.45 and 0.55, about 10 would include 9 to 11, about 1000 would include 900 to 1100.

The term “substantially” when used in connection with a geometric construction and/or geometric relationship is intended to convey that the structure so defined is nominally the geometric construction and/or geometric relationship. As one example, a portion of a chamber body that is described as being “substantially annular” is intended to convey that, although an annular (e.g., ring-shape) of the portion is desirable, some variance can occur in a “substantially annular” portion. Such variances can result from manufacturing tolerances, or other practical considerations (such as, for example, the pressure or force applied to the chamber body). Thus, a geometric construction modified by the term “substantially” includes such geometric properties within a tolerance, for example, of plus or minus 10% of the stated geometric construction.

### Chamber Body

The chamber body may include, comprise, and/or consist of any biocompatible material(s), such as one or more biocompatible polymers. The biocompatible polymer(s) may be a linear polymer, a branched polymer, a cross-linked polymer, a network polymer, and/or the like. For example, the biocompatible polymer(s) may include, comprise, and/or consist of poly(lactides), poly(glycolides), poly(lactide-co-glycolides), poly(lactic acid)s, poly(glycolic acid)s, polycarbonates, polyesteramides, polyanhydrides, polyorthoesters, poly(dioxanone)s, polycaprolactones, polyurethanes, polycyanoacrylates, and blends thereof and copolymers thereof. Examples of biocompatible polymers may include poly-(lactide-co-glycolide) (PLGA), poloxamer, polyvinylpyrrolidone (povidone or PVP), PVP ethylcellulose, sodium pyrrolidone carboxylate, poly(ethylene glycol) (PEG), poly(vinyl alcohol) (PVA), poly-(D,L-lactide-co-glycolide), poly(N-isopropyl acrylamide) (PIPA), poly(lactic acid) (PLLA or PLA), PEG-PLA, polyvinylchloride (PVC), polytetrafluroethylene (PTFE), polyethersulfone (PES), polyethylene (PE), polyetheretherketone (PEEK), polysulfone (PS), polypropylene (PP), poly(methyl methacrylate) (PMMA), poly(N-isopropyl acrylamide) (NIPAAM), gelatin, collagen, starch, or blends thereof or copolymers thereof. In some particular embodiments, the chamber body comprises and/or is formed at least in part from PMMA. In further embodiments, the chamber body comprises and/or is formed from pure PMMA. In still other embodiments, the chamber body comprises and/or is formed from a polycarbonate and/or a substantially pure polycarbonate.

In some embodiments, the biodiffusion chamber is substantially free of impurities or additives including but not limited to, for example, anti-oxidants, coloring agents, curing agents, and/or plasticizers. In some embodiments, the chamber body comprises less than 5%, 3%, 1%, 0.75%, 0.5%, 0.25%, 0.1%, 0.05%, or 0.01% of impurities or additives. In some embodiments, the chamber body comprises greater than 0.0001% and less than 5%, 3%, 1%, 0.75%, 0.5%, 0.25%, 0.1%, 0.05%, or 0.01% of impurities or additives. In particular embodiments, the chamber body comprises greater than 0.0001% and less than 1% of impurities or additives.

In some embodiments, the chamber body includes and/or comprises an opacifier. The opacifier may include, comprise, and/or consist of, for example, titanium dioxide; milk glass; mica crystals; fluorides of aluminum, calcium, barium, and magnesium; tin oxide; zirconia; zinc oxide; barium; and/or tungsten. In some embodiments, the chamber body includes and/or comprises less than 5%, 3%, 1%, 0.75%, 0.5%, 0.25%, 0.1%, 0.05%, 0.01%, or less of the opacifier. In some embodiments, the chamber body includes and/or comprises greater than 0.0001% and less than 5%, 3%, 1%, 0.75%, 0.5%, 0.25%, 0.1%, 0.05%, or 0.01% of the opacifier. In particular embodiments, the chamber body includes and/or comprises greater than 0.0001% and less than 1% of the opacifier.

The chamber body may have any shape, such as a spherical shape, a cylindrical shape, an annular or ring shape, a rectangular shape, a square shape, a polygonal shape, or any other suitable shape. In some particular embodiments, the chamber body has a ring shape. In further embodiments, the chamber body is substantially ring-shaped with the exception of a portion extending out from the chamber body—or a surface or side of the chamber body—to facilitate removal of the biodiffusion chamber from a subject (e.g., from the human body). The portion extending out from the chamber body may form and/or comprise a flange, a tab, a clip, a loop, a hook-structure or other grasping structure, or combinations thereof. In some embodiments, the portion extending our from the chamber body may define a hole, aperture, opening, slot, and/or void. In particular embodiments, the portion extending out from the chamber body and/or the hole, aperture, opening, slot, and/or void defined thereby, may facilitate removal of the biodiffusion chamber from the subject.

In some embodiments, the chamber body includes and/or comprises a first surface and a second surface. In some embodiments, the first surface (e.g., a top surface) and the second surface (e.g., a bottom surface) are substantially parallel. In some embodiments, the distance between the first surface and the second surface is between about 3.0 millimeters (mm) and about 10.0 mm. For example, the distance between the first surface and the second surface may be about 3.0 mm, 4.0 mm, 5.0 mm, 6.0 mm, 7.0 mm, 8.0 mm, 9.0 mm, or 10.0 mm, or any suitable fraction therebetween. In certain embodiments, the distance between the first surface and the second surface is about 4.5 mm. In some embodiments, at least one of the first surface and the second surface includes, comprises, and/or defines an indentation. In some embodiments, both the first surface and the second surface include, comprise, and/or define an indentation. In some embodiments, the first surface and/or the second surface may have any suitable and/or desirable surface finish configured to facilitate use and/or performance of the biodiffusion chamber. For example, in some embodiments, the first and/or second surface (or any other surface of the chamber body) may have a rough, pitted, porous, scored, and/or otherwise non-smooth surface configured to facilitate and/or enhance a coupling of the semi-permeable membranes to the first and second surfaces (e.g., can enhance friction and/or adhesion between the semi-permeable membranes and the first and second surfaces).

In some embodiments, at least one of the first surface and the second surface define at least one groove configured to facilitate the coupling and/or attachment of a first semi-permeable membrane to the first surface and/or a second semi-permeable membrane to the second surface. In some embodiments, the groove(s) can facilitate one or more manufacturing processes, steps, and/or methods. In some embodiments, the groove of the first surface and the groove of the second surface can receive a portion of a first retainer and a portion of a second retainer, respectively, which may facilitate the coupling the first retainer and the second retainer to the first surface and the second surface, respectively. The first retainer and the second retainer, in turn, retain and/or couple the first and second semi-permeable membranes to the first and second surfaces, respectively, of the chamber body.

The chamber body may define a hollow cavity. In some embodiments, the hollow cavity is substantially cylindrical having a diameter between about 5.0 mm and about 20.0 mm. For example, the diameter of the hollow cavity may be about 5.0 mm, 6.0 mm, 7.0 mm, 8.0 mm, 9.0 mm, 10.0 mm, 11.0 mm, 12.0 mm, 13.0 mm, 14.0 mm, 15.0 mm, 16.0 mm, 17.0 mm, 18.0 mm, 19.0 mm, or 20.0 mm, or any suitable fraction therebetween. In certain embodiments, the hollow cavity has a diameter of about 10.0 mm. In other embodiments, the hollow cavity may be any suitable shape, size, and/or configuration. For example, the hollow cavity may be spherical, elliptical, square, rectangular, polygonal, trapezoidal, and/or any suitable irregular shape.

In some embodiments, the hollow cavity is configured to hold a predetermined volume of fluid. In some embodiments, the hollow cavity has a volume of 100.0 microliters (μL) to 1.0 mL. In some particular embodiments, the hollow cavity has a volume of 300.0 μL to 400.0 μL. In other particular embodiments, the hollow cavity has a volume of 340.0 μL to 360.0 μL. In certain embodiments, the hollow cavity has a volume of about 350.0 μL. In some embodiments, the hollow cavity has a volume in a range of about 100.0 μL to about 10.0 mL.

In some embodiments, the hollow cavity holds or is configured to hold a composition. In some embodiments, the composition comprises at least one biologic factor. In some embodiments, the composition comprises cells, such as tumor cells. In some embodiments, the composition comprises one or more antisense molecules. In some embodiments, the composition comprises a mixture of cells and an antisense molecule. In some embodiments, the composition comprises one or more proteins. The proteins may be selected from, for example, the group consisting of enzymes, immune mediators, cytokines, growth factors, antibodies, antigens, signaling proteins, structural proteins, and fragments thereof. In some embodiments, the composition comprises cellular components (for example, microvesicles such as exosomes), microRNAs, or peptides. In some embodiments, the composition comprises one or more small molecule drugs, such as agonists or antagonists of one or more signaling or immune pathways (e.g., toll-like receptor agonists).

In some embodiments, the chamber body further includes, comprises, and/or defines an injection port. The injection port may extend from an outer surface to an inner surface of the chamber body. The injection port is in fluid communication with the hollow cavity and may be used to inject a fluid and/or a composition comprising at least one biologic factor (e.g., a composition comprising a mixture of cells, antisense molecules, buffer, and/or any other suitable biologic factor, small molecule drug, and/or the like) into the hollow cavity. In some embodiments, the injection port is a hole extending through, for example, a sidewall of the chamber body. In some embodiments, the injection port is a hole having a diameter of about 0.3 mm-8.0 mm. In some embodiments, the injection port is a hole having a diameter of about 1.0 mm to about 8.0 mm. For example, the injection port may have a diameter of about 1.0, about 2.0, about 3.0, about 4.0, about 5.0, about 6.0, about 7.0, or about 8.0 mm, or any suitable fraction therebetween. In some embodiments, the injection port is a hole having a diameter of about 0.3 mm to about 1.0 mm. For example, the injection port may have a diameter of about 0.3, about 0.4, about 0.5, about 0.6, about 0.7, about 0.8, about 0.9, or about 1.0 mm. In some embodiments, the injection port is a hole having a diameter of about 1.0 mm to about 2.0 mm. For example, the injection port may have a diameter of about 1.0 mm, about 1.1 mm, about 1.2 mm, about 1.3 mm, about 1.4 mm, about 1.5 mm, about 1.6 mm, about 1.7 mm, about 1.8 mm, about 1.9 mm, or about 2.0 mm. In particular embodiments, the injection port is a hole having a diameter of about 1.7 mm. In some embodiments, the injection port is a hole having a diameter that is at least partially based on a size of a pipette, pipette tip, and/or any other suitable device configured to convey material, product, and/or factors into the hollow cavity defined by the chamber body. In other embodiments, the injection port may be any suitable port, valve, semi-permeable member or membrane, and/or the like.

In some embodiments, the injection port is sealed or at least temporarily sealed after injection of a fluid or a composition including at least a biologic factor into the hollow cavity. In some embodiments, the injection port is reversibly or irreversibly sealed. In some embodiments, the injection port is sealed using PMMA, rubber, beeswax, paraffin, or mixtures thereof. In some embodiments, the injection port is sealed using bone wax (e.g., a sterile mixture of beeswax, paraffin, and isopropyl palmitate). In some embodiments, the injection port is sealed via a seal member, stopper, plunger, plug, and/or the like formed of and/or including any suitable material(s).

In some embodiments, the chamber body is formed of or from a single piece (e.g., a single work piece or single formed component). In some embodiments, the chamber body is a single molded structure. In some embodiments, the chamber body is a single polymeric molded structure. In some embodiments, the chamber body is assembled by coupling more than one piece, such as 2, 3, 4, 5, 6, 7, 8, 9, 10, 11, 12 pieces, or more. In some embodiments, each piece is coupled to another piece using medical grade glue, ultrasonic welding, and/or the like.

The chamber body may be formed according to several different methods. In some embodiments, the chamber is formed using 3D printing. In some embodiments, the chamber body may be formed by injection molding. 3D printing methods and injection molding methods are known to those of skill in the art.

### Removal Element

The biodiffusion chambers of the instant disclosure may further include, comprise, define, and/or otherwise be coupled to one or more elements and/or features adapted for removing the biodiffusion chamber from a subject (e.g., an animal, mammal, human, mouse, etc.). In some embodiments, the element and/or feature adapted for removing the biodiffusion chamber comprises a loop, hook, suture, flange, tab, clip, hole, or other grasping structure, or combinations thereof, that is/are coupled to or part of the chamber body. In some embodiments, such an element and/or feature is coupled to the biodiffusion chamber body through a hole or opening in the chamber body. For example, a suture or string may be threaded or inserted through and optionally tied through the hole or opening. The element and/or feature may function to allow the user to grasp the biodiffusion chamber by the element and/or feature (e.g., the loop, hook, suture, flange, tab, clip, or other grasping structure), pull upon it, and thus remove the biodiffusion chamber from the site of implantation (e.g., a portion of the human body such as the abdomen).

In some embodiments, the element and/or feature adapted for removing the biodiffusion chamber comprises and/or defines a hole or opening in the chamber body that extends from or through the first surface and to or through the second surface of the chamber body. In some embodiments, the hole or opening has a diameter of about 1.0 mm to about 1.0 cm. For example, the hole may have a diameter of 1.0 mm, 2.0 mm, 3.0 mm, 4.0 mm, 5.0 mm, 6.0 mm, 7.0 mm, 8.0 mm, 9.0 mm, or 1.0 cm, or any suitable fraction therebetween. In some embodiments, the hole or opening diameter ranges from about 4.0 mm to about 6.0 mm. In other embodiments, the hole or opening has a diameter of about 0.1 mm to about 0.9 mm. For example, the hole or opening may have a diameter of 0.1 mm, 0.2 mm, 0.3 mm, 0.4 mm, 0.5 mm, 0.6 mm, 0.7 mm, 0.8 mm, or 0.9 mm. In some embodiments, the hole or opening can have a semi-circular or irregular cross-sectional shape (e.g., taken along a plane parallel to the first surface and/or the second surface of the chamber body). In some such embodiments, the hole or opening can have a cross-sectional area of between about 1.0 mm to about 1.0 cm. For example, the hole or opening can have a cross-sectional area of about 1.0 mm², 2.0 mm², 3.0 mm², 4.0 mm², 5.0 mm², 6.0 mm², 7.0 mm², 8.0 mm², 9.0 mm², or 1.0 mm², or any suitable fraction therebetween. In certain embodiments, the hole or opening can have a cross-sectional area of about 8.0 mm².

In some embodiments, the element and/or feature adapted for removing the biodiffusion chamber includes and/or comprises a flange coupled to and/or extending from a surface of the chamber body. In some embodiments, the flange projects from a side or sidewall of the chamber body. In some embodiments, the height of the flange is less than the height of the biodiffusion chamber. In some embodiments, the height of the flange is 10%, 20%, 30%, 40%, 50%, 60%, 70%, 80%, or 90% the height of the biodiffusion chamber. In some embodiments, the flange comprises and/or defines a hole that extends between a first surface of the flange and a second surface of the flange. In some embodiments, the hole extends in a substantially perpendicular direction relative to at least one of the first surface or the second surface of the flange. In some embodiments, the flange comprises and/or defines a hole that extends transversely from or through a first side surface of the flange and to or through a second side surface of the flange. In some embodiments, the hole has a diameter of about 1.0 mm to about 1.0 cm. For example, the hole may have a diameter of about 1.0 mm, 2.0 mm, 3.0 mm, 4.0 mm, 5.0 mm, 6.0 mm, 7.0 mm, 8.0 mm, 9.0 mm, or 1.0 cm, or any suitable fraction therebetween. In some embodiments, the hole diameter ranges from about 4.0 mm to about 6.0 mm. In other embodiments, the hole has a diameter of 0.1 mm to 0.9 mm. For example, the hole may have a diameter of 0.1 mm, 0.2 mm, 0.3 mm, 0.4 mm, 0.5 mm, 0.6 mm, 0.7 mm, 0.8 mm, or 0.9 mm. In some embodiments, the hole or opening can have a semi-circular or irregular cross-sectional shape (e.g., taken along a plane parallel to the first surface and/or the second surface of the chamber body). In some such embodiments, the hole or opening can have a cross-sectional area of between about 1.0 mm to about 1.0 cm. For example, the hole or opening can have a cross-sectional area of about 1.0 mm², 2.0 mm², 3.0 mm², 4.0 mm², 5.0 mm², 6.0 mm², 7.0 mm², 8.0 mm², 9.0 mm², or 1.0 mm², or any suitable fraction therebetween. In certain embodiments, the hole or opening can have a cross-sectional area of about 8.0 mm². In some embodiments, the flange is tapered in its dimensions as it extends in a direction transverse to a longitudinal axis of the chamber body or flange and/or in a direction parallel to the longitudinal axis of the chamber body and/or flange.

In some embodiments, the element and/or feature adapted for removing the biodiffusion chamber is a suture that is threaded or inserted through a hole in the chamber body or a hole in the flange. In some embodiments, the suture is a 2-0 vicryl suture. In some instances, this design feature allows retrieval of deeper biodiffusion chamber implants (e.g., relative to previous implementations and/or implementations without such a design feature) by grasping the suture tail, allowing the surgeon to grasp the biodiffusion chambers without utilizing (e.g. Bonney or Adson) forceps. In some instances, this design feature may reduce a likelihood of puncturing one or more of the membranes during removal, which in turn, may result in undesirable portions of the composition (e.g., cells) leaking out of the biodiffusion chamber and into the subject. In some instances, this design feature may allow multiple biodiffusion chambers to be strung or at least temporarily coupled together, which facilitates removal from the subject and/or may limit a likelihood of one or more biodiffusion chambers being left in the subject during and/or after the removal process.

The element and/or feature adapted for removing the biodiffusion chamber may be located anywhere on the chamber body that facilitates removal of the biodiffusion chamber from the subject (e.g., the human body). For example, the element and/or feature adapted for removing the biodiffusion chamber may be located on the first surface, on the second surface, or on a side surface of the chamber body (e.g., a surface other than the first or second surfaces). In some embodiments, the element and/or feature adapted for removing the biodiffusion chamber is located adjacent to an injection port of the chamber body. In some embodiments, the element and/or feature adapted for removing the biodiffusion chamber is located remotely and/or otherwise spaced apart from an injection port. In some embodiments, the chamber body is substantially ring-shaped and the element and/or feature adapted for removing the biodiffusion chamber is located remotely from an injection port (e.g., at approximately 90°, 180°, or 270°, and/or any other suitable angle from the injection port). In some embodiments, the chamber body is substantially ring-shaped, the element and/or feature adapted for removing the biodiffusion chamber is and/or defines a hole in the chamber body that extends from the first surface to the second surface of the chamber body, and the element and/or feature adapted for removing the biodiffusion chamber is located across from an injection port (e.g., at approximately 180° from the injection port). In some embodiments, the chamber body is substantially ring-shaped, the element and/or feature adapted for removing the biodiffusion chamber is a flange comprising and/or defining a hole that extends through the flange from the first surface to the second surface of the flange, and the element and/or feature adapted for removing the biodiffusion chamber is located across from an injection port of the chamber body (e.g., at approximately 180° from the injection port). In other embodiments, the chamber body may have any suitable shape, the element and/or feature adapted for removing the biodiffusion chamber is a flange comprising and/or defining a hole that extends through the flange from a first surface to a second surface of the flange, and the element and/or feature adapter for removing the biodiffusion chamber is located at or in any suitable angular position relative to an injection port of the chamber body.

### Semi-Permeable Membrane

The biodiffusion chambers of the instant disclosure include and/or comprise at least one semi-permeable membrane coupled to a surface of the chamber body. In some embodiments, the biodiffusion chambers described herein include and/or comprise a first semi-permeable membrane coupled to the first surface (e.g., a top surface) of the chamber body and a second semi-permeable membrane coupled to the second surface (e.g., a bottom surface) of the chamber body. In some embodiments, the hollow cavity of the chamber body is collectively defined by and/or contained within an inner surface of the chamber body, a surface of the first semi-permeable membrane, and a surface of the second semi-permeable membrane. In some embodiments, ingress into and/or egress out of the hollow cavity may be limited to passage through the injection port, the first semi-permeable membrane, or the second semi-permeable membrane. In some embodiments, the first semi-permeable membrane and the second semi-permeable membrane include, comprise, and/or are otherwise formed of or from the same material. In other embodiments, the first semi-permeable membrane and the second semi-permeable membrane include, comprise, and/or are otherwise formed of or from different materials. In other embodiments, a biodiffusion chamber includes a single semi-permeable membrane coupled to one of the first surface or the second surface of the chamber body. In such embodiments, the surface of the chamber body opposite the surface to which the semi-permeable membrane is coupled is a closed or solid surface (e.g., does not define an opening). Thus, in such embodiments, portions of a composition conveyed into the biodiffusion chamber are diffused via the single semi-permeable membrane.

The semi-permeable membrane(s) can include, comprise, and/or can be formed of or from any suitable plastic, PTFE (e.g., Teflon™), polyester, and/or any inert or biocompatible material. In some embodiments, such an inert material can be strong, flexible, and able to withstand chemical treatments, sterilization, and/or irradiation. In some embodiments, the semi-permeable membranes are the Durapore® membrane manufactured by MilliporeSigma.

In some embodiments, the semi-permeable membranes are porous, to permit interchange, ingress, egress, diffusion, and/or passage of select factors (e.g., pharmaceutical and/or biologic products) between the chamber and the subject (e.g., patient, animal, mammal, human, mouse, etc.) once implanted. In some embodiments, the semi-permeable membranes define pores having a diameter that allows passage of small molecules but does not allow passage of cells or other relatively large molecules (i.e., the cells or other relatively large molecules cannot leave or enter the hollow cavity defined by the chamber body). In some embodiments, the diameter of the pores allows nucleic acids and other chemicals (such as, for example, cytokines produced by cells) to diffuse out of the biodiffusion chamber, but does not allow passage of cells between the biodiffusion chamber and the subject in which the biodiffusion chamber is implanted. In some embodiments, the pores of the semi-permeable membranes have a diameter of about 0.25 μm or smaller. In certain embodiments, the pores have a diameter of not more than about 0.25 μm. In specific embodiments, the pores have a diameter of about 0.1 μm. In particular embodiments, the pores range in diameter from about 0.1 μm to about 0.25 μm. In other words, a semi-permeable membrane can include pores having different and/or varied diameters in the range of about 0.1 μm to about 0.25 μm. In some embodiments, the diameter of the pores is greater than about 0.25 μm but less than about 25 μm. For example, the diameter of the pores may be about 0.5 μm, about 0.75 μm, about 1 μm, about 2 μm, about 3 μm, about 4 μm, about 5 μm, about 6 μm, about 7 μm, about 8 μm, about 9 μm, about 10 μm, about 11 μm, about 12 μm, about 13 μm, about 14 μm, about 15 μm, about 16 μm, about 17 μm, about 18 μm, about 19 μm, about 20 μm, about 21 μm, about 22 μm, about 23 μm, or about 24 μm. In some embodiments, the diameter of the pores is about 0.25 μm to about 1 μm, about 1 μm to about 10 μm, or about 10 μm to about 25 μm.

### Method of Making a Biodiffusion Chamber

In some instances, a method of making the biodiffusion chambers of the disclosure includes providing a chamber body and one or more semi-permeable membranes. The semi-permeable membrane(s) may be cut to match the shape and/or size of the first and/or second surfaces of the chamber body. In some embodiments, the semi-permeable membrane(s) are cut to match the shape and/or size of at least the hollow cavity defined by the chamber body. In some embodiments, the semi-permeable membrane(s) are cut to fit within an indentation on or defined by the first and/or second surfaces of the chamber body. In some embodiments, the semi-permeable membrane(s) have a size or area that is larger than the first surface and/or the second surface of the chamber body and is/are cut to fit the size and/or shape of the first surface and/or the second surface once coupled thereto. Such an arrangement can allow a portion of the semi-permeable membrane(s) to lay over and/or conform to one or more features disposed on and/or defined by the first surface and/or the second surface (e.g., one or more protrusions, ridges, indentations, grooves, slots, etc.).

The semi-permeable membrane(s) can be coupled to the chamber body in any suitable manner. In some embodiments, for example, the semi-permeable membranes are coupled to the chamber body using an adhesive (e.g., medical grade glue). In some embodiments, the medical grade glue includes, comprises, and/or consists of an ethylene monomer or polymer such as the alpha cyanoacrylates, a silicone glue, or PMMA. In other embodiments, the semi-permeable membranes are coupled to the chamber body using ultrasonic welding. Methods and devices for performing ultrasonic welding are known to those of skill in the art.

In some embodiments, a first semi-permeable membrane is coupled to the first surface of the biodiffusion chamber, and a second semi-permeable membrane is coupled to a second surface of the biodiffusion chamber. In some embodiments, a first semi-permeable membrane is coupled to the first surface of the biodiffusion chamber and at least partially disposed within a first indentation, groove, slot, and/or the like, and a second semi-permeable membrane is coupled to a second surface of the biodiffusion chamber and at least partially disposed within a second indentation, groove, slot, and/or the like. In such embodiments, at least a portion of the semi-permeable membrane can be substantially flush with the first surface and at least a portion of the second semi-permeable membrane can be substantially flush with the second surface.

In some embodiments, the semi-permeable membranes are coupled to the chamber body using a mechanical fastener, coupler, clamp, retainer, compression member, and/or the like. For example, the biodiffusion chamber can include a first retainer that is fastened, coupled, and/or affixed to the first surface of the chamber body and a second retainer that is fastened, coupled, and/or affixed to the second surface of the chamber body. In such embodiments, the first semi-permeable membrane is disposed on and/or placed in contact with at least a portion of the first surface and then the first retainer is fastened, coupled, and/or affixed to the first surface (e.g., via an adhesive, ultrasonic welding, an interference or snap fit, a threaded coupling, etc.) such that the first semi-permeable membrane is disposed between the first surface and the first retainer. Likewise, the second semi-permeable membrane is disposed on and/or placed in contact with at least a portion of the second surface and then the second retainer is fastened, coupled, and/or affixed to the second surface such that the second semi-permeable membrane is disposed between the second surface and the second retainer. Thus, the first retainer and the second retainer can be configured to retain the first semi-permeable membrane and the second semi-permeable membrane, respectively, in a fixed position relative to the first surface and the second surface, respectively, of the chamber body. In other words, the first retainer and the second retainer can be coupled to the chamber body to fixedly couple the first semi-permeable membrane and the second semi-permeable membrane to the chamber body.

In some embodiments, the semi-permeable membranes are coupled to the chamber body using a combination of techniques and/or methods such as, for example, a mechanical fastener or clamping device and ultrasonic welding. In some implementations, the first and second semi-permeable membranes can be coupled to the chamber body in substantially the same manner. In other implementations the first semi-permeable membrane can be coupled to the first surface of the chamber body via a first method or first combination of methods, and the second semi-permeable membrane can be coupled to the second surface of the chamber body via a second method or second combination of methods (e.g., different from the first method or first combination of methods).

### Formulation and Use of the Biodiffusion Chamber

The biodiffusion chambers of the disclosure are adapted for insertion into and removal from a subject. In some embodiments, the subject is an animal. In some embodiments, the subject is a mammal. In some embodiments, the subject is a human. In some embodiments, the subject is a mouse.

In some embodiments, one or more biodiffusion chambers is implanted into a site of interest within a subject. In some embodiments, the site of interest is a diseased site. In some embodiments, the site of interest is remote, separate, and/or spaced apart from a diseased site.

In some embodiments, one or more biodiffusion chambers is/are implanted surgically into a subject. In some embodiments, one or more biodiffusion chambers is/are implanted into the subject's abdomen. In certain embodiments, one or more biodiffusion chambers is/are implanted into the rectus sheath of a subject. In some instances, 1-50 chambers are implanted into a subject. For example 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 biodiffusion chambers are implanted into a subject.

In some embodiments, the one or more biodiffusion chambers are removed from the subject after a therapeutically effective amount of time. In some instances, the therapeutically effective amount of time can be between about 3 hours and about 72 hours. In some instances, the therapeutically effective amount of time is about 3 hours, about 6 hours, about 12 hours, about 24 hours, about 36 hours, about 48 hours, about 60 hours, about 72 hours, or more or any time or faction of time therebetween. In some embodiments, the therapeutically effective amount of time is greater than about 72 hours, greater than about 96 hours, greater than about 1 week, greater than about 1 month, greater than about 3 months, greater than about 6 months, or greater than about 1 year. In some embodiments, the chambers are implanted in the subject indefinitely, or until the subject has an adverse response to the chamber. Typically, the chambers are implanted for about 15 hours to about 30 hours, or from about 24 hours to about 72 hours. In some particular implementations, 20 chambers are implanted for about 40 hours to about 50 hours.

The biodiffusion chambers of the disclosure may be used for many purposes, including but not limited to systemic and local drug delivery, gene therapy, autologous cell vaccination, and/or the like. Various compositions of materials may be inserted into and/or at least temporarily contained within the hollow cavity of the biodiffusion chamber before implantation into a subject. Such compositions may include at least one or more biologic factors. For example, a composition may include and/or may be a mixture of cell(s), antisense molecule(s), buffer(s), and/or any other suitable biologic factor. In some implementations, such compositions can includes a mixture of cell(s), multiple different antisense molecules, buffer(s), small drug molecules, and/or any other suitable material, product, drug, or factor. Such compositions may be inserted into the hollow cavity of the biodiffusion chamber through the injection port (described above). After all materials of interest have been added to the biodiffusion chamber, and before implantation of the biodiffusion chamber into a subject, the injection port may be sealed. In some embodiments, the injection port is sealed using PMMA, rubber, beeswax, or paraffin, or mixtures thereof. In some embodiments, the injection port is sealed using bone wax.

In some embodiments, the injection port is sealed using a stopper, plug, plunger, insert, and/or occlusion member. Such a stopper or the like can include and/or can be formed of PMMA, rubber, silicone and/or any suitable biocompatible material configured to elastically deform. In some embodiments, such a stopper or the like can be configured to form a substantially fluid tight seal with at least one surface of the chamber body defining at least a portion of the injection port. In some embodiments, such a stopper or the like can be fixedly and/or non-removably inserted into the injection port after the materials of interest have been added to the biodiffusion chamber. In other embodiments, such a stopper or the like can be removably inserted into the injection port after the materials of interest have been added to the biodiffusion chamber.

In some embodiments, a composition including at least a therapeutically effective amount of an antisense molecule is inserted and/or conveyed into the biodiffusion chamber before implantation. In some embodiments, the therapeutically effective amount of the antisense molecule is about 1.0 microgram (μg) to about 5.0 μg. For example, the therapeutically effective amount may be about 1.0 μg, about 2.0 μg, about 3.0 μg, about 4.0 μg, about 5.0 μg, about 6.0 μg, about 7.0 μg, about 8.0 μg, about 9.0 μg, or about 10.0 μg. In some embodiments, the therapeutically effective amount of the antisense molecule is about 5.0 μg to about 50.0 μg. In some embodiments, the therapeutically effective amount of the antisense molecule is about 50.0 μg to about 100.0 μg. In some embodiments, the therapeutically effective amount of the antisense molecule is about 10.0 μg to about 500.0 μg. In some embodiments, the therapeutically effective amount of the antisense molecule is about 100.0 μg to about 500.0 μg. In some embodiments, the therapeutically effective amount of the antisense molecule is about 500.0 μg to about 1.0 milligram (mg). In some embodiments, the therapeutically effective amount of the antisense molecule is about 1.0 mg to about 3.0 mg. In some embodiments, the therapeutically effective amount of the antisense molecule is about 3.0 mg to about 5.0 mg. In some embodiments, the therapeutically effective amount of the antisense molecule is about 5.0 mg to about 10.0 mg. In some embodiments, the therapeutically effective amount of the antisense molecule is about 1.0 μg to about 10.0 mg.

In some embodiments, the antisense molecule is an antisense oligodeoxynucleotide (AS-ODN). In some embodiments, the antisense molecule comprises a modified phosphate backbone. In some embodiments, the phosphate backbone modification renders the antisense more resistant to nuclease degradation. In certain embodiments, the modification is a locked antisense. In other embodiments, the modification is a phosphorothioate linkage. In certain embodiments, the antisense contains one or more phosphorothioate linkages. In certain embodiments, the phosphorothioate linkages stabilize the antisense molecule by conferring nuclease resistance, thereby increasing its half-life. In some embodiments, the antisense may be partially phosphorothioate-linked. For example, up to about 1%, up to about 3%, up to about 5%, up to about 10%, up to about 20%, up to about 30%, up to about 40%, up to about 50% up to about 60%, up to about 70%, up to about 80%, up to about 90%, up to about 95%, or up to about 99% (or any percentage or fraction of a percent therebetween) of the antisense may be phosphorothioate-linked. In some embodiments, the antisense is fully phosphorothioate-linked. In other embodiments, phosphorothioate linkages may alternate with phosphodiester linkages. In certain embodiments, the antisense has at least one terminal phosphorothioate monophosphate.

In some embodiments, the antisense molecule comprises one or more CpG motifs. In other embodiments, the antisense molecule does not comprise a CpG motif. In certain aspects, the one or more CpG motifs are methylated. In other aspects, the one or more CpG motifs are unmethylated. In certain embodiments, the one or more unmethylated CpG motifs elicit an innate immune response when the antisense molecule is administered to a subject.

In certain embodiments, the antisense molecule comprises at least one terminal modification or “cap”. The cap may be a 5′ and/or a 3′-cap structure. As used herein, the terms “cap” or “end-cap” include chemical modifications at either terminus of the oligonucleotide (with respect to terminal ribonucleotides), and including modifications at the linkage between the last two nucleotides on the 5′ end and the last two nucleotides on the 3′ end. The cap structure may increase resistance of the antisense molecule to exonucleases without compromising molecular interactions with the target sequence or cellular machinery. Such modifications may be selected on the basis of their increased potency in vitro or in vivo. The cap can be present at the 5′-terminus (5′-cap) or at the 3′-terminus (3′-cap) or can be present on both ends. In certain embodiments, the 5′- and/or 3′-cap is independently selected from phosphorothioate monophosphate, abasic residue (moiety), phosphorothioate linkage, 4′-thio nucleotide, carbocyclic nucleotide, phosphorodithioate linkage, inverted nucleotide or inverted abasic moiety (2′-3′ or 3′-3′), phosphorodithioate monophosphate, and methylphosphonate moiety. The phosphorothioate or phosphorodithioate linkage(s), when part of a cap structure, are generally positioned between the two terminal nucleotides on the 5′ end and the two terminal nucleotides on the 3′ end.

In some embodiments, the antisense molecule may also comprise one or more p-ethoxy backbone modifications as disclosed in U.S. Pat. No. 9,744,187, filed Oct. 14, 2016, the disclosure of which is incorporated herein by reference in its entirety. In some embodiments, the nucleic acid backbone of the antisense molecule comprises at least one p-ethoxy backbone linkage. For example, up to about 1%, up to about 3%, up to about 5%, up to about 10%, up to about 20%, up to about 30%, up to about 40%, up to about 50% up to about 60%, up to about 70%, up to about 80%, up to about 90%, up to about 95%, or up to about 99% of the antisense molecule may be p-ethoxy-linked.

In some embodiments, the antisense molecule targets the expression of Insulin like Growth Factor 1 Receptor (IGF-1R). IGF-1R is a tyrosine kinase cell surface receptor that shares 70% homology with the insulin receptor. When activated by its ligands (IGF-I, IGF-II, and insulin), it regulates broad cellular functions including proliferation, transformation and cell survival. The IGF-1R plays a role during growth in anchorage-independent conditions that may occur in malignant tissues.

In certain embodiments, the antisense molecule is directed against DNA or RNA of a growth factor or growth factor receptor, such as, for example, IGF-IR.

In certain embodiments, the antisense is a deoxynucleotide directed against IGF-1R (IGF-1R AS ODN). The full-length coding sequence of IGF-1R (SEQ ID NO: 1) is provided in, for example, WIPO Patent Publication No. WO 2016/164916, filed Apr. 11, 2016, the disclosure of which is incorporated herein by reference in its entirety.

In certain embodiments, the IGF-1R AS ODN comprises nucleotide sequences complementary to the IGF-1R signal sequence, comprising either RNA or DNA. The signal sequence of IGF-1R is a 30 amino acid sequence. In other embodiments, the IGF-1R AS ODN comprises nucleotide sequences complementary to portions of the IGF-1R signal sequence, comprising either RNA or DNA. In some embodiments, the IGF-1R AS ODN comprises nucleotide sequences complementary to codons 1-309 of IGF-1R, comprising either RNA or DNA. In other embodiments, the IGF-1R AS ODN comprises nucleotide sequences complementary to portions of codons 1-309 of IGF-1R, comprising either RNA or DNA.

In certain embodiments, the IGF-1R AS ODN is at least about 5 nucleotides, at least about 10 nucleotides, at least about 15 nucleotides, at least about 20 nucleotides, at least about 25 nucleotides, at least about 30 nucleotides, at least about 35 nucleotides, at least about 40 nucleotides, at least about 45 nucleotides, or at least about 50 nucleotides in length. In some embodiments, the IGF-1R AS ODN is from about 15 nucleotides to about 22 nucleotides in length. In certain embodiments, the IGF-1R AS ODN is about 18 nucleotides in length.

In some aspects, the IGF-1R AS ODN comprises the nucleotide sequence 5′-TCCTCCGGAGCCAGACTT-3′ (SEQ ID NO: 2), or a fragment thereof. In certain embodiments, the IGF-1R AS ODN may have at least about 70%, at least about 75%, at least about 80%, at least about 85%, at least about 90%, at least about 95%, at least about 96%, at least about 98%, or 100% identity to SEQ ID NO: 2. In some embodiments, the IGF-1R AS ODN comprises one or more phosphorothioate linkages. In certain embodiments, the IGF-1R AS ODN consists of the nucleotide sequence of SEQ ID NO: 2.

NOBEL is an 18-mer oligodeoxynucleotide with a phosphorothioate backbone and a sequence complimentary to codons 2 through 7 in the IGF-1R gene. As such, NOBEL is an antisense oligonucleotide directed against IGF-1R (IGF-1R AS ODN). The NOBEL sequence, derived as the complimentary sequence of the IGF-1R gene at the 5′ end, is: 5′-TCCTCCGGAGCCAGACTT-3′ (SEQ ID NO: 2). NOBEL has a stable shelf life and is resistant to nuclease degradation due to its phosphorothioate backbone.

Suitable antisense nucleic acids are also described in U.S. Patent Publication No. 2017/0056430, filed Apr. 11, 2016, the disclosure of which is incorporated herein by reference in its entirety. In some embodiments, a composition conveyed into the biodiffusion chamber may include multiple different types, kinds, and/or forms of antisense molecules.

In some embodiments, cells may be inserted into the biodiffusion chamber. In some embodiments, the cells are cancer cells. In some embodiments, the cells are isolated or derived from a solid tumor. In some embodiments, the cancer cells are glioma cells. In some embodiments, the cancer cells are isolated or derived from astrocytoma, hepatocarcinoma, breast cancer, head and neck squamous cell cancer, lung cancer, renal cell carcinoma, hepatocellular carcinoma, gall bladder cancer, classical Hodgkin's lymphoma, esophageal cancer, uterine cancer, rectal cancer, thyroid cancer, melanoma, colorectal cancer, prostate cancer, ovarian cancer, and pancreatic cancer. In some embodiments, the cancer cells are isolated or derived from the subject into which the biodiffusion chamber is implanted.

In some embodiments, a therapeutically effective number of cells may be inserted into the chamber. The therapeutically effective number of tumor cells may be, for example, about 7.5×10⁵ to about 1.25×10⁶ cells per chamber. In some embodiments, the therapeutically effective number of tumor cells is about 1.0×10⁶ cells per chamber.

In some embodiments, a pharmaceutically acceptable carrier or excipient is inserted into the biodiffusion chamber. In some embodiments, a buffer is inserted into the biodiffusion chamber. In some embodiments, the buffer is saline.

In some embodiments, a composition comprising a therapeutically effective amount of an antisense molecule and a therapeutically effective number of cells (e.g., glioma cells) is inserted into the chamber. In some embodiments, a composition comprising a therapeutically effective amount of an antisense molecule, a therapeutically effective number of cells (e.g., glioma cells), and a pharmaceutically acceptable carrier is inserted into the chamber. In some embodiments, the antisense molecule has the sequence of SEQ ID NO: 1.

In some embodiments, a composition comprising at least (i) a therapeutically effective amount of one or more antisense molecules, (ii) a therapeutically effective number of cells (e.g., glioma cells), and (iii) one or more additional materials such as buffer(s), small molecule drugs, additional biologic factors, and/or the like is inserted and/or conveyed into the biodiffusion chamber. Further formulations and uses for biodiffusion chambers are disclosed in WIPO Patent Publication No. WO2018/165528, filed Mar. 9, 2018, the disclosure of which is incorporated by reference herein in its entirety.

The biodiffusion chambers of the instant disclosure may be used to prevent or treat a disease or condition in a subject in need thereof. In some embodiments, the chambers are used to treat or prevent a cancer, including those selected from the group consisting of glioma, astrocytoma, hepatocarcinoma, breast cancer, head and neck squamous cell cancer, lung cancer, renal cell carcinoma, hepatocellular carcinoma, gall bladder cancer, classical Hodgkin's lymphoma, esophageal cancer, uterine cancer, rectal cancer, thyroid cancer, melanoma, colorectal cancer, prostate cancer, ovarian cancer, and pancreatic cancer. In specific embodiments, the cancer is a glioma. In certain aspects, the glioma is recurrent malignant glioma. In some embodiments, the cancer is an astrocytoma. In certain embodiments, the subject who is a candidate for treatment is suffering from WHO grade II, WHO grade III, or WHO grade IV tumor. In some aspects, the tumor is an astrocytoma. In certain embodiments, the tumor is selected from grade II astrocytoma, AIII (IDH1 R132H mutant grade III astrocytoma), AIII-G (IDH1 wild-type grade III with characteristics of glioblastoma multiforme astrocytoma), or grade IV astrocytoma.

In some embodiments, a method of treating cancer in a patient comprises administering to the patient a biodiffusion chamber of the disclosure. In some embodiments, the patient suffers from a solid tumor. In some embodiments, the patient suffers from glioma. In some embodiment, the patient suffers from a cancer selected from the group consisting of astrocytoma, hepatocarcinoma, breast cancer, head and neck squamous cell cancer, lung cancer, renal cell carcinoma, hepatocellular carcinoma, gall bladder cancer, classical Hodgkin's lymphoma, esophageal cancer, uterine cancer, rectal cancer, thyroid cancer, melanoma, colorectal cancer, prostate cancer, ovarian cancer, and pancreatic cancer. In some embodiments, the biodiffusion chamber comprises an antisense molecule, tumor cells, a buffer, and optionally, an additional agent. In some embodiments, the administering comprises surgically implanting the biodiffusion chamber for a therapeutically effective period of time (e.g., about 3 to about 72 hours).

In some embodiments, the biodiffusion chambers of the disclosure are used in any of the methods of treating cancer described in US 2017/0056430 or US 2018/0256625, which are incorporated by reference herein in their entireties.

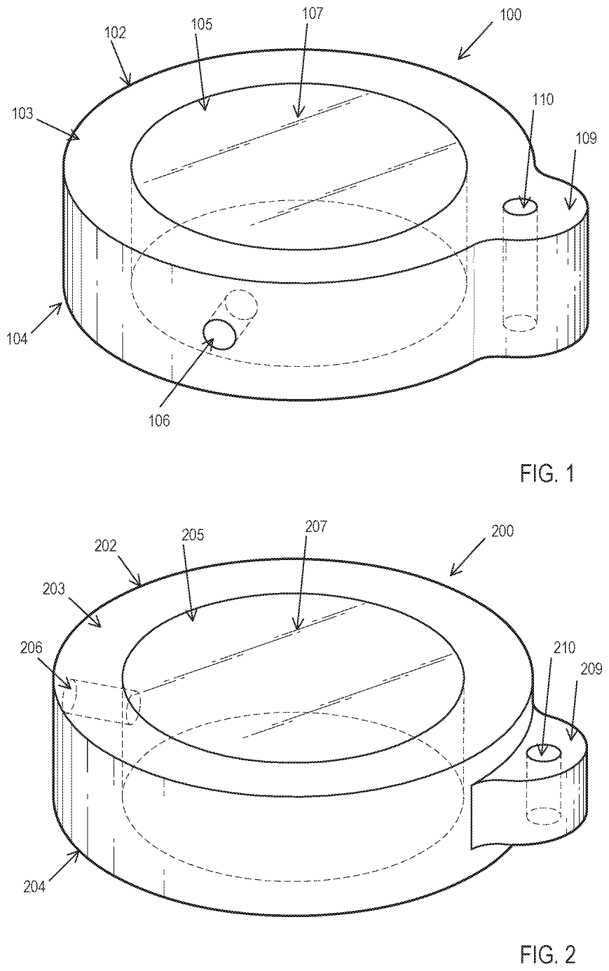

FIG. 1 depicts a biodiffusion chamber 100, according to an embodiment. As seen therein, the biodiffusion chamber 100 includes and/or comprises a chamber body 102 that is substantially annular (e.g., ring-shaped) except for a portion 109 that extends from an exterior surface or side of the ring-shaped structure. The chamber body 102 has a first surface 103 (e.g., a top surface) and a second surface 104 (e.g., a bottom surface). The distance between the first surface 103 and the second surface 104 is about 4.0 mm.

The chamber body 102 includes an interior surface that defines a cylindrically-shaped hollow cavity 105. The walls of the chamber body 102 between the exterior surface and the interior surface are about 2.0 mm thick. The hollow cavity 105 has a diameter of approximately 10.0 mm, a height of approximately 4.0 mm, and a volume of approximately 315.0 μL.

The chamber body 102 further comprises and/or defines an injection port 106, which extends through the walls of the chamber body 102 from the exterior surface of the chamber body 102 to the interior surface of the chamber body 102. The diameter of the injection port 106 is approximately 5.0 mm.

A first semi-permeable membrane 107 is coupled to the first surface 103 of the chamber body 102, and a second semi-permeable membrane (not shown) is coupled to the second surface 104 of the chamber body 102. The first semi-permeable membrane 107 and the second semi-permeable membrane can be coupled to the first surface 103 and the second surface 104, respectively, via ultrasonic welding and/or an adhesive such as medical grade glue.

The biodiffusion chamber 101 further includes, comprises, and/or defines an element and/or feature for removing the biodiffusion chamber 101 from a subject. As shown, the element and/or feature is a hole 110 within the portion 109 that extends from the exterior surface or side of the ring-shaped structure. The hole 110 extends from the first surface 103 of the chamber body 102 to the second surface 104 of the chamber body 102. The hole 110 is substantially perpendicular to at least one of the first surface 103 or the second surface 104. A suture (not shown) may optionally be threaded and/or inserted through the hole 110. The diameter of the hole 110 is approximately 5.0 mm. The hole 110 is located remotely from (e.g., approximately 90° from) the injection port 106.

FIG. 2 depicts a biodiffusion chamber 200, according to an embodiment. As seen therein, the biodiffusion chamber 200 includes and/or comprises a chamber body 202 that is substantially annular (e.g., ring-shaped) except for a flange 209 that extends from an exterior surface or side of the ring-shaped structure. The chamber body 202 has a first surface 203 (e.g., a top surface) and a second surface 204 (e.g., a bottom surface). The distance between the first surface 203 and the second surface 204 is about 4.0 mm.

The chamber body 202 includes an interior surface that defines a cylindrically-shaped hollow cavity 205. The walls of the chamber body 202 between the exterior surface and the interior surface are about 2.0 mm thick. The hollow cavity 205 has a diameter of approximately 10.0 mm, a height of approximately 4.0 mm, and a volume of approximately 315.0 μL.

The chamber body 202 further comprises and/or defines an injection port 206, which extends through the walls of the chamber body 202 from the exterior surface of the chamber body 202 to the interior surface of the chamber body 202. The diameter of the injection port 206 is approximately 5.0 mm.

A first semi-permeable membrane 207 is coupled to the first surface 203 of the chamber body 202, and a second semi-permeable membrane (not shown) is coupled to the second surface 204 of the chamber body 202. The first semi-permeable membrane 207 and the second semi-permeable membrane can be coupled to the first surface 203 and the second surface 204, respectively, via ultrasonic welding and/or an adhesive such as medical grade glue.

The flange 209 facilitates removal of the biodiffusion chamber 201 from a subject. The flange 209 has a height less than the height of the chamber body 202 (e.g., less than about 4.0 mm). The flange 209 comprises and/or defines a hole 210 that extends from a first surface of the flange 209 (e.g., a top surface) to a second surface of the flange 209 (e.g., a bottom surface). The hole 210 is substantially perpendicular to at least one of the first surface of the flange 209 or the second surface of the flange 209. The diameter of the hole 210 is approximately 5.0 mm. A suture (not shown) may optionally be threaded through the hole 210. The flange 209 is located across from (e.g., approximately 180° from) and/or opposite the injection port 206.

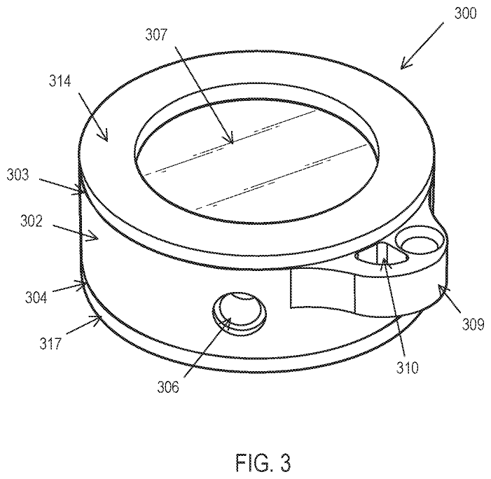

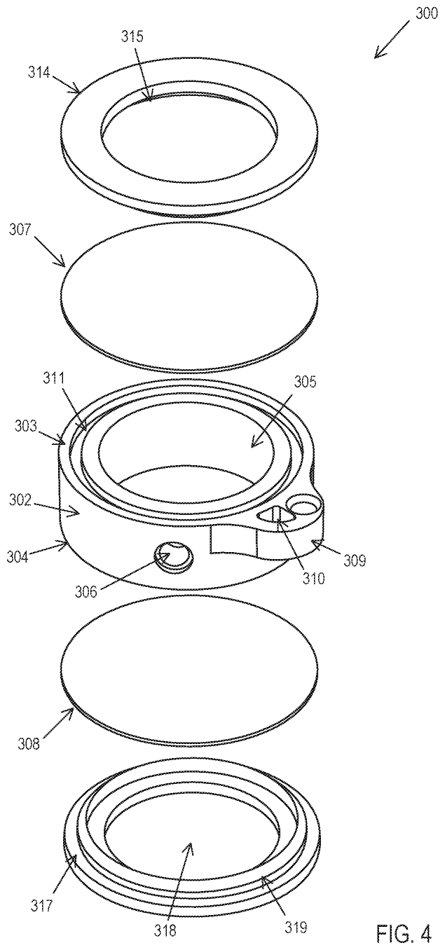

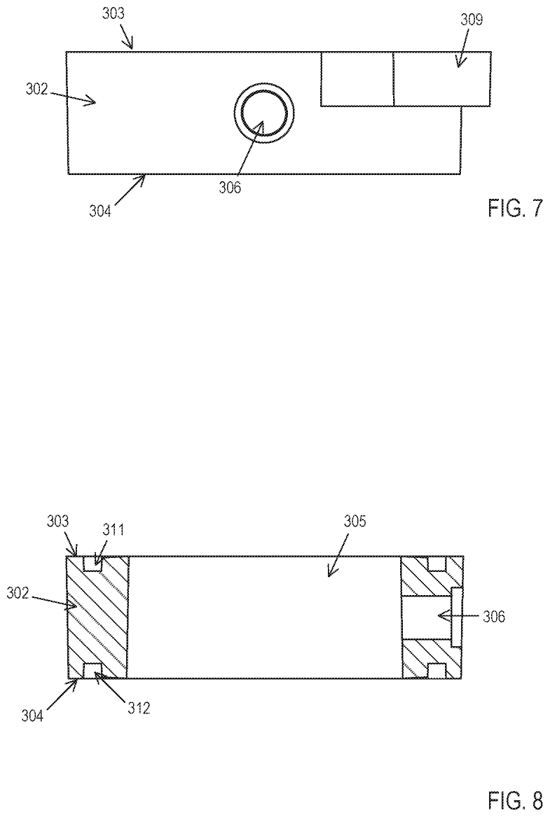

FIGS. 3-15 depict a biodiffusion chamber 300, according to an embodiment. As shown in FIGS. 3 and 4, the biodiffusion chamber 300 includes and/or comprises a chamber body 302, a first semi-permeable membrane 307, a second semi-permeable membrane 308, a first retainer 314, and a second retainer 317. The biodiffusion chamber 300 is configured to at least temporarily contain a composition including at least a biologic factor (e.g., a composition including a mixture of cells, antisense molecules, a buffer, and/or any other additional agents such as small molecule drugs, additional and/or different antisense molecule(s), additional and/or different buffer(s), and/or the like). Moreover, the biodiffusion chamber 300 is configured to be inserted into a subject (e.g., an animal, mammal, human, and/or mouse) and removed from the subject after a predetermined time (e.g., a therapeutically effective time of about 3 hours to about 72 hours, as described above).

As shown in FIGS. 4-6, the chamber body 302 has a first surface 303 (e.g., a top surface) and a second surface 304 (e.g., a bottom surface). In some embodiments, the distance between the first surface 303 and the second surface 304 is about 4.0 mm. In other embodiments, the distance between the first surface 303 and the second surface 304 is any suitable distance (e.g., less than 4.0 mm or greater than 4.0 mm). The chamber body 302 has a substantially cylindrical perimeter except for a flange 309 that extends from and/or that is coupled to an exterior surface or sidewall of the cylindrical perimeter of the chamber body 302. More specifically, the chamber body 302 is substantially annular (e.g., ring-shaped) with an interior surface that defines a cylindrically-shaped hollow cavity 305. The walls of the chamber body 302 between the exterior surface and the interior surface are about 2.0 mm thick. The hollow cavity 305 has a diameter of approximately 10.0 mm, a height of approximately 4.5 mm, and a volume of approximately 350.0 μL. In other embodiments, the hollow cavity 305 can have any suitable size, shape, and/or configuration.

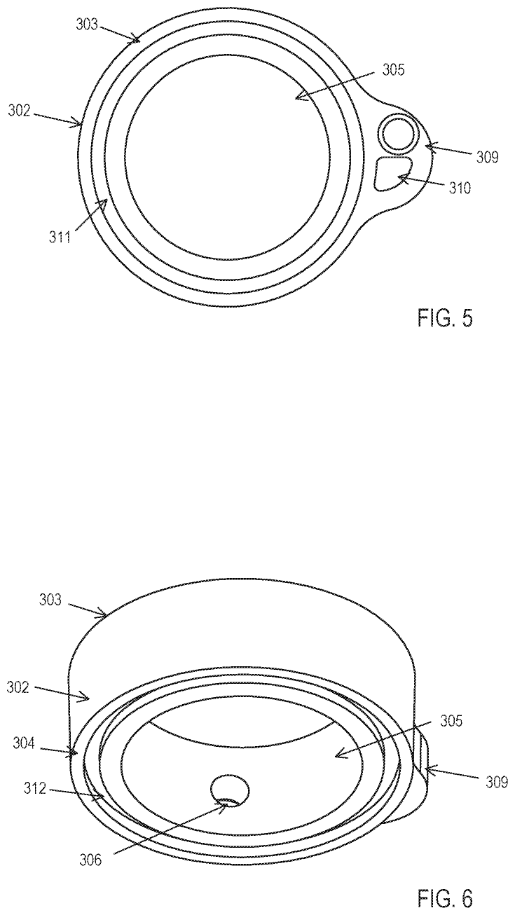

The first surface 303 defines a groove 310 that substantially surrounds, encircles, encompasses, and/or circumscribes the hollow cavity 305 (see e.g., FIGS. 4 and 5). Similarly, the second surface 304 defines a groove 311 that substantially surrounds, encircles, encompasses, and/or circumscribes the hollow cavity 305 (see e.g., FIG. 6). In some embodiments, the groove 310 of the first surface 303 and the groove 311 of the second surface 304 can facilitate one or more manufacturing processes, steps, and/or methods. In some embodiments, the groove 310 of the first surface 303 and the groove 311 of the second surface 304 can facilitate the coupling, securement, and/or attachment of the first semi-permeable membrane 307 to the first surface 303 and the coupling, securement, and/or attachment of the second semi-permeable membrane 308 to the second surface 304.

As shown in FIGS. 4-7, the flange 309 has a height less than the height of the chamber body 302 (e.g., less than about 4.0 mm). The flange 309 comprises and/or defines a hole 310 that extends from a first surface of the flange 309 (e.g., a top surface) to a second surface of the flange 309 (e.g., a bottom surface). The hole 310 is substantially perpendicular to at least one of the first surface of the flange 309 or the second surface of the flange 309. The diameter of the hole 310 is approximately 5.0 mm.

As shown in FIGS. 6-8, the chamber body 302 defines an injection port 306 that is in fluid communication with the hollow cavity 305. The injection port 306 extends through a wall of the chamber body 302 from the exterior surface of the chamber body 302 to the interior surface of the chamber body 302 (FIG. 8). In some embodiments, the diameter of the injection port 306 is approximately 5.0 mm. In other embodiments, the diameter of the injection port 306 is based on a size of a pipette or pipette tip used to convey fluid and/or a composition including at least one or more biologic factors into the hollow cavity 305.

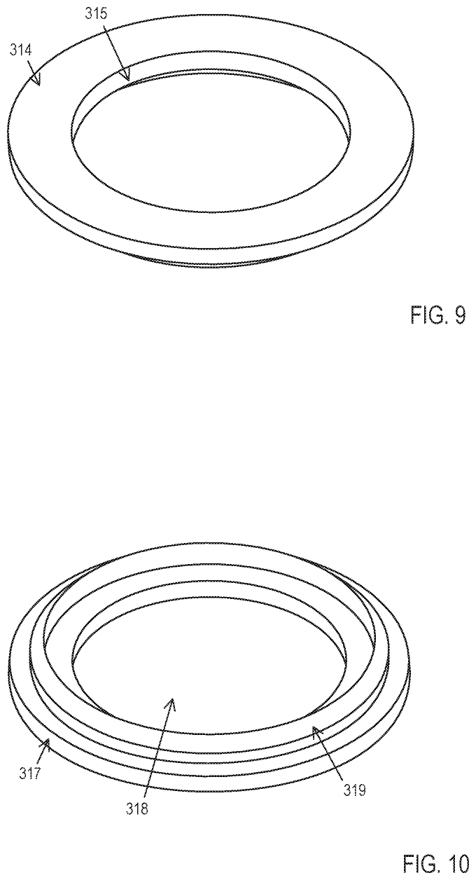

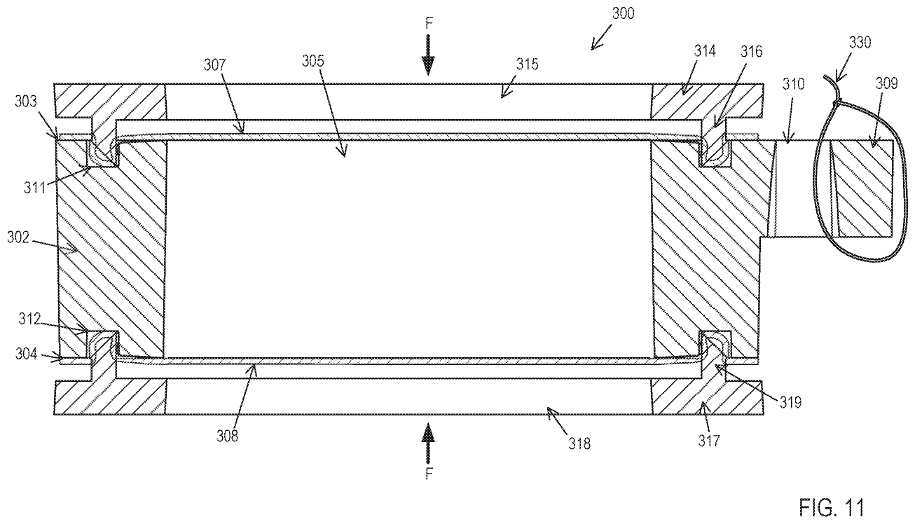

As shown in FIG. 9, the first retainer 314 of the biodiffusion chamber 300 is substantially annular or ring-shaped and defines an opening 315. The first retainer 314 has a size and shape that is substantially similar to the size and shape of the first surface 303 of the chamber body 302 (e.g., excluding the flange 309 that may be coplanar to or with the first surface 303). The opening 315 of the first retainer 314 has a size and shape (perimeter) that is substantially similar to the size and shape (perimeter) of the hollow cavity 305. The first retainer 314 also includes a protrusion 316 configured to facilitate the coupling of the first retainer 314 to the first surface 303 of the chamber body 302, as described below with reference to FIG. 11.

As shown in FIG. 10, the second retainer 317 of the biodiffusion chamber 300 is substantially annular or ring-shaped and defines an opening 318. The second retainer 317 has a size and shape that is substantially similar to the size and shape of the second surface 304 of the chamber body 302. The opening 318 of the second retainer 317 has a size and shape (perimeter) that is substantially similar to the size and shape (perimeter) of the hollow cavity 305. The second retainer 317 also includes a protrusion 319 configured to facilitate the coupling of the second retainer 317 to the second surface 304 of the chamber body 302, as described below with reference to FIG. 11.

The first retainer 314 and the second retainer 317 are configured to couple to the first surface 303 and the second surface 304, respectively. In addition, the coupling of the first retainer 314 to the first surface 303 and the coupling of the second retainer 317 to the second surface 304 is operable in securing the first semi-permeable membrane 307 to the first surface 303 and the second semi-permeable membrane 308 to the second surface 304, respectively, as shown in FIG. 11.

More specifically, during manufacturing, the first semi-permeable membrane 307 can be placed in contact with and/or otherwise disposed on the first surface 303 of the chamber body 302. A portion of the first semi-permeable membrane 307 can overlay and/or can otherwise be disposed over or in the groove 311 defined by the first surface 303. As shown in FIG. 11, the first retainer 314 can be aligned with the first surface 303 and positioned on a portion of the first semi-permeable membrane 307. Similarly, the second semi-permeable membrane 308 can be placed in contact with and/or otherwise disposed on the second surface 304 of the chamber body 302. A portion of the second semi-permeable membrane 308 can overlay and/or can otherwise be disposed over or in the groove 312 defined by the second surface 304. As shown in FIG. 11, the second retainer 317 can be aligned with the second surface 304 and positioned on a portion of the second semi-permeable membrane 308.

When the first retainer 314 and/or the second retainer 317 are in a desirable position, ultrasonic energy can be applied on at least one of the first retainer 314 or the second retainer 317 to couple the first retainer 314 to the first surface 303 and/or to couple the second retainer 317 to the second surface 304. As shown in FIG. 11, applying and/or transferring ultrasonic energy can result in a force F exerted on at least one of the first retainer 314 and/or the second retainer 317. Moreover, the first retainer 314 and the second retainer 317 can be aligned relative to the chamber body 302 such that the protrusion 316 of the first retainer 314, and in turn, a portion of the first semi-permeable membrane 307 are pushed into the groove 311 defined by the first surface 303. The force F (i.e., the ultrasonic energy) can result in at least a portion of the protrusion 316 melting and/or otherwise deforming within the groove 311, thereby fixedly coupling the first retainer 314 to the first surface 303. In addition, the first retainer 314 and the first surface 303 clamp a portion of the first semi-permeable membrane 307 disposed therebetween to fixedly secure or couple the first semi-permeable membrane 307 to the first surface 303. The second retainer 317 and the second semi-permeable protrusion 316 of the first retainer 314 can contact a portion of the first semi-permeable membrane 308 are fixedly coupled to the second surface 304 in substantially the same manner. In some instances, the first retainer 314 and the second retainer 317 are coupled to the first surface 303 and the second surface 304, respectively, at the same time, and/or in substantially the same manufacturing process. In other instances, the first retainer 314 is coupled to the first surface 303 independent of the coupling of the second retainer 317 to the second surface 304.

As described above, the chamber body 302 defines an injection port 306 that is in fluid communication with the hollow cavity 305 and used to convey a desired amount of fluid and/or a desired amount of a composition including at least one or more biologic factors into the hollow cavity 305. In some implementations, the injection port 306 is sealed after conveying a desired amount of fluid and/or composition into the hollow cavity 305.

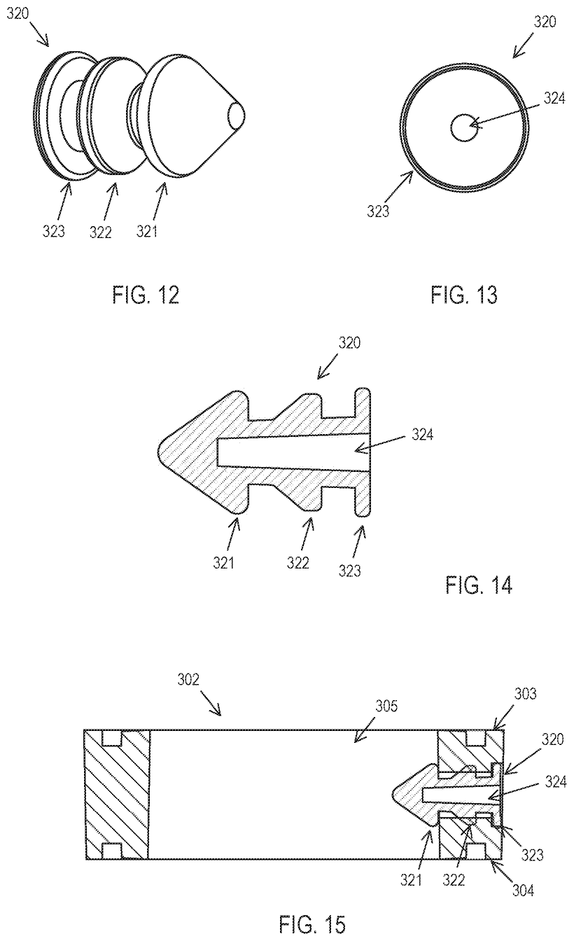

For example, as shown in FIGS. 12-15, the injection port 306 can be transitioned from first or open state to a second or closed state in response to a plug 320 being inserted into the injection port 306. In some embodiments, the plug 320 can include and/or can be formed of PMMA, rubber, silicon, and/or any suitable biocompatible material configured to elastically deform. The plug 320 includes a first seal 321, a second seal 322, and a third seal 323, which are each configured to form a substantially fluid tight seal with at least one surface of the chamber body 302 defining at least a portion of the injection port 306 and/or the hollow cavity 305. For example, in some embodiments, when the plug 320 is inserted into the injection port 306, the second seal 322 can form an interference or friction fit with an inner surface of the chamber body 302 defining the injection port 306, which in turn, results in a substantially fluid tight seal therebetween, as shown in FIG. 15. Moreover, when the plug 320 is inserted into the injection port 306, the first seal 321 can be disposed in the hollow cavity 305 and in contact with the interior surface of the chamber body 302 that defines the hollow cavity 305, which in turn, results in a substantially fluid tight seal therebetween, as shown in FIG. 15. In addition, the arrangement of the first seal 321 can be operable in fixedly coupling the plug 320 to the chamber body 302 when the plug 320 is inserted into the injection port 306.

Although not shown in FIGS. 3-15, in some embodiments, an insertion tool can be used to insert the plug 320 into the injection port 306. For example, in some embodiments, the insertion tool can include a rod configured to be inserted into an opening 324 defined by the plug 320 and can further include a shoulder that is in contact with an exterior surface of the plug 320 when the rod is inserted into the opening 324. In some instances, a user can insert the rod into the opening 324 of the plug 320 and can exert a force of the insertion tool to insert the plug 320 into the injection port 306. Once the plug 320 is disposed in the injection port 306, the insertion tool may be removed, leaving the plug 320 within—and thereby, sealing—the injection port 306.

Referring back to FIG. 11, in some implementations, the flange 309 and/or the opening 310 defined by the flange 309 facilitates removal of the biodiffusion chamber 300 from a subject (e.g., an animal, mammal, and/or human). For example, in some embodiments, a suture 330 may be threaded and/or inserted through the hole 310 and coupled to the flange 309. The suture 330 can be an element and/or feature adapted for removing the biodiffusion chamber 300. In some instances, the biodiffusion chamber 300 can be inserted into a subject with the suture 330 coupled to the flange 309. After a predetermined time (e.g., 3 hours to 72 hours after insertion into the subject), the suture 330 can be engaged and manipulated to remove the biodiffusion chamber 300 from the subject.

Although not shown in FIGS. 3-15, in some implementations, a suture (e.g., the suture 330) can be inserted through an opening defined by any number of biodiffusion chambers. In such implementations, the suture (e.g., the suture 330) can at least temporarily couple or string together the number of biodiffusion chambers. Moreover, in some instances, coupling and/or stringing together multiple biodiffusion chambers can facilitate removal of the entire string of biodiffusion chambers.

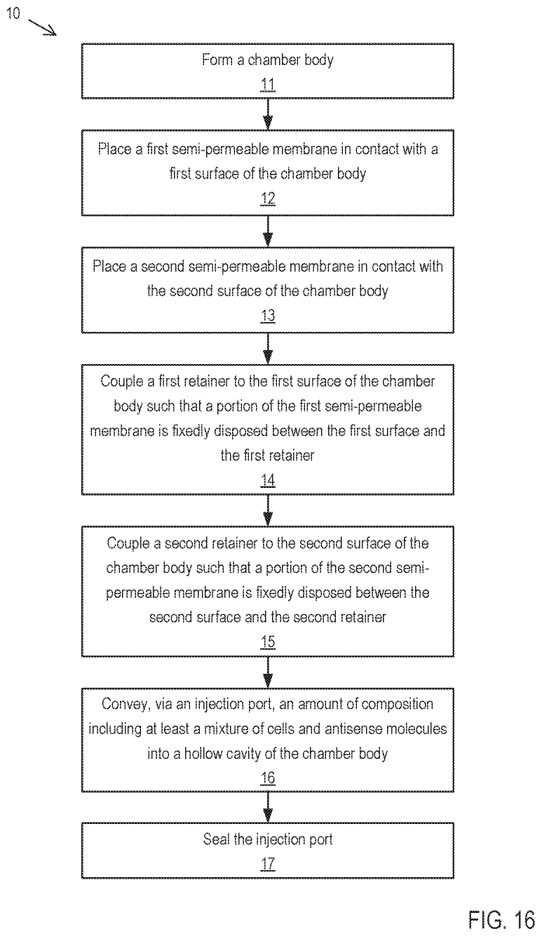

FIG. 16 is a flowchart depicting a method 10 of manufacturing a biodiffusion chamber, such as those described herein, according to an embodiment. The method 10 includes forming a chamber body of the biodiffusion chamber, at 11. As described in detail above, the chamber body can be formed from any suitable biocompatible material via injection molding, 3D printing, and/or any other suitable method. The chamber body can be similar to, for example, the chamber body 302 described above with reference to FIGS. 3-15. Accordingly, the chamber body defines a hollow cavity and an injection port in fluid communication with the hollow cavity, and has a first surface, a second surface, and a flange that defines an opening.

A first semi-permeable membrane is placed in contact with the first surface of the chamber body, at 12. A second semi-permeable membrane is placed in contact with the second surface of the chamber body, at 13. The first semi-permeable membrane and the second semi-permeable membrane can be substantially similar to, for example, the first semi-permeable membrane 307 and the second semi-permeable membrane 308 described above with reference to FIGS. 3-15. Accordingly, the semi-permeable membranes can be permeable to relatively small molecules such as nucleic acids, antisense molecules, cytokines, and/or other chemicals but impermeable to relatively large molecules such as cells.

A first retainer is coupled to the first surface of the chamber body such that a portion of the first semi-permeable membrane is fixedly disposed between the first surface and the first retainer, at 14. For example, as described above with reference to the biodiffusion chamber 300, the first retainer can be fixedly coupled to the first surface via ultrasonic welding and/or any other suitable method. With the portion of the first semi-permeable membrane disposed between the first surface and the first retainer, coupling the first retainer to the first surface, in turn, couples the first semi-permeable membrane to the first surface.

A second retainer is coupled to the second surface of the chamber body such that a portion of the second semi-permeable membrane is fixedly disposed between the second surface and the second retainer, at 15. For example, as described above with reference to the biodiffusion chamber 300, the second retainer can be fixedly coupled to the second surface via ultrasonic welding and/or any other suitable method. With the portion of the second semi-permeable membrane disposed between the second surface and the second retainer, coupling the second retainer to the second surface, in turn, couples the second semi-permeable membrane to the second surface.

Coupling the first and second retainers to the first and second surfaces, respectively, results in the first and second semi-permeable membrane being coupled to the first and second surfaces, respectively. Thus, after coupling the first and second retainers to the first and second surfaces, respectively, an amount of a composition including at least a mixture of cells and antisense molecules is conveyed, via an injection port, into a hollow cavity defined by the chamber body, at 16. The injection port can be substantially similar to, for example, the injection port 306 described above with reference to the biodiffusion chamber 300. As described above, the arrangement of the semi-permeable membranes can be such that the first and second semi-permeable membranes are permeable to the antisense molecule but not to larger molecules. Accordingly, in some instances, inserting the biodiffusion chamber into a subject (e.g., an animal, mammal, human, mouse, etc.) can allow an amount of the antisense molecule to diffuse out of the biodiffusion chamber and into the subject.

After conveying the antisense molecule, the injection port is sealed, at 17. The injection port can be sealed in any suitable manner such as those described herein. For example, in some embodiments, a plug can be inserted into the injection port to seal the injection port and/or to otherwise transition the injection port from a first or open state to a second or sealed state, as described above with reference to the plug 320.

As described above, in a first implementation, a biodiffusion chamber is adapted for insertion into and removal from the human body, wherein the biodiffusion chamber comprises (a) a chamber body defining a hollow cavity, and including a first surface and a second surface; (b) a first semi-permeable membrane coupled to the first surface; (c) a second semi-permeable membrane coupled to the second surface; and (d) an element adapted for removing the biodiffusion chamber from the human body; wherein the first semi-permeable membrane and the second semi-permeable membrane are permeable to fluids and soluble factors but are not permeable to cells.

In a second implementation, a biodiffusion chamber is adapted for insertion into and removal from the human body, wherein the biodiffusion chamber comprises (a) a chamber body defining a hollow cavity, and including a first surface and a second surface; (b) a first semi-permeable membrane coupled to the first surface; (c) a second semi-permeable membrane coupled to the second surface; and (d) a hole in the chamber body that extends vertically from the first surface to the second surface of the chamber body and that facilitates removal of the biodiffusion chamber from the human body; wherein the first semi-permeable membrane and the second semi-permeable membrane are permeable to fluids and soluble factors but are not permeable to cells.

In a third implementation, a biodiffusion chamber is adapted for insertion into and removal from the human body, wherein the biodiffusion chamber comprises (a) a chamber body defining a hollow cavity, and including a first surface and a second surface; (b) a first semi-permeable membrane coupled to the first surface; and (c) a second semi-permeable membrane coupled to the second surface; wherein the chamber body is substantially ring shaped with the exception of a portion extending out from the chamber body that defines a hole, wherein the hole extends vertically from the first surface to the second surface of the chamber body and facilitates removal of the biodiffusion chamber from the human body; wherein the chamber body is a single molded structure; and wherein the first semi-permeable membrane and the second semi-permeable membrane are permeable to fluids and soluble factors but are not permeable to cells.

In a fourth implementation, a biodiffusion chamber is adapted for insertion into and removal from the human body, wherein the biodiffusion chamber comprises (a) a chamber body defining a hollow cavity, and including a first surface and a second surface; (b) a first semi-permeable membrane coupled to the first surface; and (c) a second semi-permeable membrane coupled to the second surface; wherein the chamber body is substantially ring shaped with the exception of an portion extending out from the chamber body comprising a flange, wherein the flange comprises a hole that extends vertically from a first surface of the flange to a second surface of the flange and facilitates removal of the biodiffusion chamber from the human body; wherein the chamber body is a single molded structure; and wherein the first semi-permeable membrane and the second semi-permeable membrane are permeable to fluids and soluble factors but are not permeable to cells.

In some embodiments, the biodiffusion chamber according to at least one of the first implementation, the second implementation, the third implementation, and/or the fourth implementation can further include, where applicable, one or more of the following elements, features, and/or aspects:
the element adapted for removing the biodiffusion chamber from the human body comprises a suture coupled to the chamber body;
the element adapted for removing the biodiffusion chamber from the human body comprises a hole in the chamber body;
a suture is threaded through the hole in the chamber body;
the element adapted for removing the biodiffusion chamber from the human body comprises a flange coupled to and extending from the chamber body;
the flange comprises a hole that extends from a first surface to a second surface of the flange;
the distance from the first surface to the second surface of the flange is less than the distance from the first surface to the second surface of the chamber body;
a suture is threaded through the hole in the flange;
the chamber body is a single polymeric molded structure;
the chamber body is substantially ring shaped;
the diameter of the hollow cavity is 5.0 mm-20.0 mm;
the diameter of the hollow cavity is 10.0 mm;
the distance between the first surface and the second surface of the chamber body is 3.0 mm-10.0 mm;
the distance between the first surface and the second surface of the chamber body is 4.0 mm;
the diameter of the hole in the chamber body is 3.0 mm-8.0 mm;
the diameter of the hole in the chamber body is 5.0 mm;
a suture is threaded through the hole in the chamber body;
the chamber body comprises poly(methyl methacrylate);
the chamber body comprises pure poly(methyl methacrylate);
the chamber body is substantially free of anti-oxidants, coloring agents, curing agents, and plasticizers;
the chamber body comprises less than 0.1% impurities or additives;
the chamber body comprises an opacifier;
the chamber body comprises less than 1% of the opacifier;
the opacifier is titanium dioxide;
the first semi-permeable membrane is coupled to the first surface and the second semi-permeable membrane is coupled to the second surface using ultrasonic welding;
the first semi-permeable membrane is coupled to the first surface and the second semi-permeable membrane is coupled to the second surface using a medical grade glue;
the medical grade glue comprises poly(methyl methacrylate) (PMMA);
the chamber body further comprises a first indentation on the first surface and a second indentation on the second surface;
the first semi-permeable membrane is coupled to the first surface within the first indentation and the second semi-permeable membrane is coupled to the second surface within the second indentation, the first semi-permeable membrane is substantially flush with the first surface and the second semi-permeable membrane is substantially flush with the second surface;
the chamber body further comprises an injection port;
the injection port is a hole having a diameter of 3.0 mm-8.0 mm;
the injection port hole has a diameter of 5.0 mm;
the injection port is sealed;
the injection port is sealed using bone wax;
the injection port is located remotely from the element adapted for removing the biodiffusion chamber from the human body;
the injection port is located remotely from the hole in the chamber body;
the chamber body is substantially ring shaped, and wherein the injection port is located approximately 180° from the element adapted for removing the biodiffusion chamber from the human body;
the chamber body is substantially ring shaped, and wherein the injection port is located approximately 180° from the hole in the chamber body;
the hollow cavity has a volume of 100.0 μL to 1.0 mL;
the hollow cavity has a volume of 310.0 μL to 320.0 μL;
the biodiffusion chamber further comprises a therapeutically effective amount of an antisense molecule;
the antisense molecule is an antisense oligodeoxynucleotide;
the antisense molecule comprises at least one phosophorothioate linkage;
the antisense molecule has the sequence 5′-TCCTCCGGAGCCAGACTT-3′ (SEQ ID NO: 2);
the therapeutically effective amount is 1.0 μg to 5.0 μg;
the therapeutically effective amount is 1.0 μg to 10.0 mg; and
the therapeutically effective amount is 2.0 μg.

In some embodiments, a method of making the biodiffusion chamber according to at least one of the first implementation, the second implementation, the third implementation, and/or the fourth implementation can include and/or can comprise one or more of the following steps, elements, features, and/or aspects:
forming the chamber body using injection molding;
forming the chamber body using 3D printing;
coupling the first semi-permeable membrane to the first surface and the second semi-permeable membrane to the second surface using ultrasonic welding;
coupling the first semi-permeable membrane to the first surface and the second semi-permeable membrane to the second surface using medical grade glue; and
coupling the first semi-permeable membrane to the first surface and the second semi-permeable membrane to the second surface using medical grade glue comprising poly(methyl methacrylate) (PMMA).

While various embodiments have been described above, it should be understood that they have been presented by way of example only, and not limitation. Where schematics and/or embodiments described above indicate certain components arranged in certain orientations or positions, the arrangement of components may be modified. While the embodiments have been particularly shown and described, it will be understood that various changes in form and details may be made. For example, while the biodiffusion chamber 300 is shown and described above as including the first semi-permeable membrane 307 and the second semi-permeable membrane 308, in other embodiments, a biodiffusion chamber can include a single semi-permeable membrane. In such embodiments, the semi-permeable membrane may be coupled to a first surface of a chamber body while a second surface of the chamber body opposite the first surface is closed, sealed, solid, and/or otherwise lacking an opening, hole, port, or the like.

Although various embodiments have been described as having particular features and/or combinations of components, other embodiments are possible having a combination of any features and/or components from any of embodiments described herein. For example, while the 209 of the chamber body 202 is shown in FIG. 2 and described above as defining the hole 210 that is substantially cylindrical (e.g., a circular cross-sectional shape), in other embodiments, the flange 209 of the chamber body 202 can define a hole or opening that has a shape similar to the hole or opening 310 defined by the flange 309 (see e.g., FIG. 5), or vice versa.

The specific configurations of the various components can also be varied. For example, the size and specific shape of the various components can be different from the embodiments shown, while still providing the functions as described herein. More specifically, the size and shape of the various components can be specifically selected for a desired or intended usage. Thus, it should be understood that the size, shape, and/or arrangement of the embodiments and/or components thereof can be adapted for a given use unless the context explicitly states otherwise.

Where methods and/or events described above indicate certain events and/or procedures occurring in certain order, the ordering of certain events and/or procedures may be modified. Additionally, certain events and/or procedures may be performed concurrently in a parallel process when possible, as well as performed sequentially as described above.

All of the products, compositions, and/or methods disclosed and/or claimed herein can be made and executed without undue experimentation in light of the present disclosure. While the products, compositions, and/or methods of this disclosure have been described in terms of preferred embodiments, it will be apparent to those of skill in the art that variations and modifications may be applied to the products, compositions, and/or methods and in the steps or in the sequence of steps of the methods described herein without departing from the concept, spirit and scope of the disclosure. All such similar variations and modifications apparent to those skilled in the art are deemed to be within the spirit, scope, and concept of the disclosure as defined by the appended claims.

What is claimed:

1. A biodiffusion chamber configured for insertion into and removal from a subject, the biodiffusion chamber comprising:
a chamber body including a first surface and a second surface, and defining a hollow cavity, the chamber body configured to at least temporarily contain an amount of a composition including at least a mixture of cells and antisense molecules within the hollow cavity, a portion of the chamber body configured to be engaged by a removal member configured to enable removal of the biodiffusion chamber from the subject;
a first semi-permeable membrane configured to be coupled to the first surface; and
a second semi-permeable membrane configured to be coupled to the second surface, wherein the first semi-permeable membrane and the second semi-permeable membrane are permeable to the antisense molecules and impermeable to the cells.

2. The biodiffusion chamber of claim 1, wherein the portion of the chamber body defines an opening.

3. The biodiffusion chamber of claim 1 or claim 2, wherein the removal member is a suture.

4. The biodiffusion chamber of claim 3, wherein the suture is configured to be coupled to the portion of the chamber body.

5. The biodiffusion chamber of claim 1, wherein the removal member is a suture, the portion of the chamber body defines an opening, and the suture is configured to be inserted through the opening.

6. The biodiffusion chamber any one of claims 1-5, wherein the portion of the chamber body is a flange extending from a surface of the chamber body other than the first surface and the second surface.

7. The biodiffusion chamber of claim 1, wherein the portion of the chamber body is a flange extending from a surface of the chamber body other than the first surface and the second surface, the flange defines an opening, an axis associated with the opening being substantially parallel to an axis associated with the hollow cavity.

8. The biodiffusion chamber of claim 7, wherein the removal member is a suture, the suture configured to be inserted through the opening.

9. The biodiffusion chamber of claim 8, wherein the biodiffusion chamber is a first biodiffusion chamber from a plurality of biodiffusion chambers, a portion of the suture is configured to be inserted through an opening defined by a portion of a second biodiffusion chamber from the plurality of biodiffusion chambers to at least temporarily couple the first biodiffusion chamber from the plurality of biodiffusion chambers to the second biodiffusion chamber from the plurality of biodiffusion chambers.

10. The biodiffusion chamber of any one of claims 1-9, further comprising:
a first retainer configured to be coupled to the first surface of the chamber body such that a portion of the first semi-permeable membrane is fixedly disposed between the first retainer and the first surface of the chamber body; and
a second retainer configured to be coupled to the second surface of the chamber body such that a portion of the second semi-permeable membrane is fixedly disposed between the second retainer and the second surface of the chamber body.

11. The biodiffusion chamber of claim 10, wherein the first retainer is coupled to the first surface of the chamber body via ultrasonic welding, the second retainer is coupled to the second surface of the chamber body via ultrasonic welding.

12. The biodiffusion chamber of claim 10 or claim 11, wherein the first surface of the chamber body defines a first groove and the second surface of the chamber body defines a second groove.

13. The biodiffusion chamber of claim 12, wherein a portion of the first retainer and a portion of the first semi-permeable membrane are fixedly disposed in the first groove when the first retainer is coupled to the first surface of the chamber body, and
a portion of the second retainer and a portion of the second semi-permeable membrane are fixedly disposed in the second groove when the second retainer is coupled to the second surface of the chamber body.

14. A biodiffusion chamber configured for insertion into and removal from a subject, the biodiffusion chamber comprising:
a chamber body including a first surface, a second surface, and a flange, the flange defining an opening configured to receive at least a portion of a removal member, the chamber body defining a hollow cavity and an injection port in fluid communication with the hollow cavity, the injection port configured to convey a composition including at least an amount of a biologic factor into the hollow cavity;
a first semi-permeable membrane in contact with the first surface;
a second semi-permeable membrane in contact with the second surface;
a first retainer fixedly coupled to the first surface such that a portion of the first semi-permeable membrane is disposed between the first retainer and the first surface; and
a second retainer fixedly coupled to the second surface such that a portion of the second semi-permeable membrane is disposed between the second retainer and the second surface,
the first semi-permeable membrane and the second semi-permeable membrane being permeable to the biologic factor and impermeable to cells.

15. The biodiffusion chamber of claim 14, wherein the first surface of the chamber body defines a first groove and the second surface of the chamber body defines a second groove.

16. The biodiffusion chamber of claim 15, wherein a portion of the first retainer and a portion of the first semi-permeable membrane are fixedly disposed in the first groove, and
a portion of the second retainer and a portion of the second semi-permeable membrane are fixedly disposed in the second groove.

17. The biodiffusion chamber of any one of claims 14-16, wherein the first retainer is coupled to the first surface of the chamber body via ultrasonic welding, and the second retainer is coupled to the second surface of the chamber body via ultrasonic welding.

18. The biodiffusion chamber of any one of claims 14-16, wherein the first retainer is coupled to the first surface of the chamber body and the second retainer is coupled to the second surface of the chamber body via an adhesive.

19. The biodiffusion chamber of any one of claims 14-18, wherein an axis associated with the opening is substantially parallel to an axis associated with the hollow cavity, and an axis associated with the injection port is substantially perpendicular to the axis associated with the opening and the axis associated with the hollow cavity.

20. The biodiffusion chamber of any one of claims 14-19, wherein the injection port has a first state and a second state, the injection port configured to convey the composition into the hollow cavity when in the first state, the injection port being sealed when in the second state.

21. The biodiffusion chamber of claim 20, further comprising:
a plug configured to be inserted into the injection port, the plug comprising at least one seal configured to form a fluid tight seal with a surface of the chamber body to place the injection port in the second state.

22. The biodiffusion chamber of any one of claims 14-21, wherein the removal member is a suture configured to be inserted into the opening defined by the flange.

23. The biodiffusion chamber of claim 22, wherein the biodiffusion chamber is a first biodiffusion chamber from a plurality of biodiffusion chambers, a portion of the suture is configured to be inserted through an opening defined by a portion of a second biodiffusion chamber from the plurality of biodiffusion chambers to at least temporarily couple the first biodiffusion chamber from the plurality of biodiffusion chambers to the second biodiffusion chamber from the plurality of biodiffusion chambers.

24. The biodiffusion chamber of any one of claims 14-23, wherein the hollow cavity has a volume between 100.0 microliters (μL) to 1.0 milliliters (mL).

25. The biodiffusion chamber of any one of claims 14-24, wherein the hollow cavity has a volume of about 350.0 μL.

26. The biodiffusion chamber of any one of claims 14-25, wherein the composition including at least the amount of the biologic factor comprises a therapeutically effective amount of an antisense molecule.

27. The biodiffusion chamber of claim 26, wherein the therapeutically effective amount of the antisense molecule is between about 1.0 micrograms (μg) to about 10.0 milligrams (mg).

28. The biodiffusion chamber of claim 27, wherein the therapeutically effective amount of the antisense molecule is about 1.0 μg, about 2.0 μg, about 3.0 μg, about 4.0 μg, about 5.0 μg, about 6.0 μg, about 7.0 μg, about 8.0 μg, about 9.0 μg, or about 10.0 μg.

29. The biodiffusion chamber of claim 26, wherein the therapeutically effective amount of the antisense molecule is about 10.0 μg to about 500.0 μg.

30. The biodiffusion chamber of any of claims 26-29, wherein the antisense molecule is an antisense oligodeoxynucleotide.

31. The biodiffusion chamber of any of claims 26-29, wherein the antisense molecule includes at least one phosophorothioate linkage.

32. The biodiffusion chamber of any of claims 26-29, wherein the antisense molecule has the sequence 5′-TCCTCCGGAGCCAGACTT-3′ (SEQ ID NO: 2).

33. The biodiffusion chamber of any of claims 14-32, wherein the composition including at least the amount of the biologic factor comprises a therapeutically effective number of tumor cells.

34. The biodiffusion chamber of claim 33, wherein the therapeutically effective number of tumor cells is about 7.5×10⁵ to about 1.25×10⁶.

35. The biodiffusion chamber of claim 33, wherein the therapeutically effective number of tumor cells is about 1.0×10⁶.

36. A method of manufacturing a biodiffusion chamber, the method comprising:
forming a chamber body, the chamber body defining a hollow cavity and an injection port in fluid communication with the hollow cavity, the chamber body having a first surface, a second surface, and a flange, the flange defining an opening;
placing a first semi-permeable membrane in contact with the first surface of the chamber body;
placing a second semi-permeable membrane in contact with the second surface of the chamber body;
coupling a first retainer to the first surface of the chamber body such that a portion of the first semi-permeable membrane is fixedly disposed between the first surface and the first retainer;
coupling a second retainer to the second surface of the chamber body such that a portion of the second semi-permeable membrane is fixedly disposed between the second surface and the second retainer;
conveying, via the injection port, a composition including a mixture of cells and an antisense molecule into the hollow cavity after the coupling of the first retainer to the first surface and the coupling of the second retainer to the second surface; and
sealing the injection port after the conveying.

37. The method of claim 36, wherein the coupling of the first retainer to the first surface includes coupling the first retainer to the first surface via ultrasonic welding such that the first semi-permeable membrane is fixedly coupled to the first surface, and
the coupling of the second retainer to the second surface includes coupling the second retainer to the second surface via ultrasonic welding such that second semi-permeable membrane is fixedly coupled to the second surface.

38. The method of claim 36 of claim 37, wherein the biodiffusion chamber is configured to be inserted into and removed from a subject.

39. The method of any of claims 36-38, wherein the opening defined by the flange is configured to receive a portion of a removal member configured to enable removal of the biodiffusion chamber from the portion of the body.

40. The method of claim 39, wherein the removal member is a suture, a portion of the suture being disposed in the opening defined by the flange.

41. The method of claim 40, wherein the biodiffusion chamber is a first biodiffusion chamber, a portion of the suture is configured to be inserted through an opening defined by a portion of a second biodiffusion chamber to at least temporarily couple the first biodiffusion chamber to the second biodiffusion chamber, the suture configured to enable removal of the first biodiffusion chamber and the second biodiffusion chamber from the portion of the body.

42. The method of any of claims 36-41, wherein the antisense molecule is an antisense oligodeoxynucleotide.

43. The method of any of claims 36-41, wherein the antisense molecule includes at least one phosophorothioate linkage.

44. The method of any of claims 36-41, wherein the antisense molecule has the sequence 5′-TCCTCCGGAGCCAGACTT-3′ (SEQ ID NO: 2).

45. The method of any of claims 36-44, wherein the conveying of the composition includes conveying between 1.0 micrograms (μg) to 10.0 milligrams (mg) of the antisense molecule into the hollow cavity.

46. The method of any of claims 36-45, wherein the sealing of the injection port after the conveying includes inserting a plug into the injection port such that at least one seal of the plug forms a fluid tight seal with a surface of the chamber body.

47. A method of treating cancer in a patient in need thereof, the method comprising administering to the patient a biodiffusion chamber of any one of claims 1 to 35.

48. The method of claim 47, wherein the patient suffers from glioma.

49. The method of claim 47, wherein the patient suffers from astrocytoma, hepatocarcinoma, breast cancer, head and neck squamous cell cancer, lung cancer, renal cell carcinoma, hepatocellular carcinoma, gall bladder cancer, classical Hodgkin's lymphoma, esophageal cancer, uterine cancer, rectal cancer, thyroid cancer, melanoma, colorectal cancer, prostate cancer, ovarian cancer, or pancreatic cancer.


SEQUENCE LISTING (2 sequences)

SEQ ID NO: 1 - DNA, 4104 bp, Homo sapiens
atgaagtctggctccggaggagggtccccgacctcgctgtgggggctcctgtttctctccgccgcgctctcgctctggccgacgagtggagaaatctgcgggccaggcatcgacatccgcaacgactatcagcagctgaagcgcctggagaactgcacggtgatcgagggctacctccacatcctgctcatctccaaggccgaggactaccgcagctaccgcttccccaagctcacggtcattaccgagtacttgctgctgttccgagtggctggcctcgagagcctcggagacctcttccccaacctcacggtcatccgcggctggaaactcttctacaactacgccctggtcatcttcgagatgaccaatctcaaggatattgggctttacaacctgaggaacattactcggggggccatcaggattgagaaaaatgctgacctctgttacctctccactgtggactggtccctgatcctggatgcggtgtccaataactacattgtggggaataagcccccaaaggaatgtggggacctgtgtccagggaccatggaggagaagccgatgtgtgagaagaccaccatcaacaatgagtacaactaccgctgctggaccacaaaccgctgccagaaaatgtgcccaagcacgtgtgggaagcgggcgtgcaccgagaacaatgagtgctgccaccccgagtgcctgggcagctgcagcgcgcctgacaacgacacggcctgtgtagcttgccgccactactactatgccggtgtctgtgtgcctgcctgcccgcccaacacctacaggtttgagggctggcgctgtgtggaccgtgacttctgcgccaacatcctcagcgccgagagcagcgactccgaggggtttgtgatccacgacggcgagtgcatgcaggagtgcccctcgggcttcatccgcaacggcagccagagcatgtactgcatcccttgtgaaggtccttgcccgaaggtctgtgaggaagaaaagaaaacaaagaccattgattctgttacttctgctcagatgctccaaggatgcaccatcttcaagggcaatttgctcattaacatccgacgggggaataacattgcttcagagctggagaacttcatggggctcatcgaggtggtgacgggctacgtgaagatccgccattctcatgccttggtctccttgtccttcctaaaaaaccttcgcctcatcctaggagaggagcagctagaagggaattactccttctacgtcctcgacaaccagaacttgcagcaactgtgggactgggaccaccgcaacctgaccatcaaagcagggaaaatgtactttgctttcaatcccaaattatgtgtttccgaaatttaccgcatggaggaagtgacggggactaaagggcgccaaagcaaaggggacataaacaccaggaacaacggggagagagcctcctgtgaaagtgacgtcctgcatttcacctccaccaccacgtcgaagaatcgcatcatcataacctggcaccggtaccggccccctgactacagggatctcatcagcttcaccgtttactacaaggaagcaccctttaagaatgtcacagagtatgatgggcaggatgcctgcggctccaacagctggaacatggtggacgtggacctcccgcccaacaaggacgtggagcccggcatcttactacatgggctgaagccctggactcagtacgccgtttacgtcaaggctgtgaccctcaccatggtggagaacgaccatatccgtggggccaagagtgagatcttgtacattcgcaccaatgcttcagttccttccattcccttggacgttctttcagcatcgaactcctcttctcagttaatcgtgaagtggaaccctccctctctgcccaacggcaacctgagttactacattgtgcgctggcagcggcagcctcaggacggctacctttaccggcacaattactgctccaaagacaaaatccccatcaggaagtatgccgacggcaccatcgacattgaggaggtcacagagaaccccaagactgaggtgtgtggtggggagaaagggccttgctgcgcctgccccaaaactgaagccgagaagcaggccgagaaggaggaggctgaataccgcaaagtctttgagaatttcctgcacaactccatcttcgtgcccagacctgaaaggaagcggagagatgtcatgcaagtggccaacaccaccatgtccagccgaagcaggaacaccacggccgcagacacctacaacatcaccgacccggaagagctggagacagagtaccctttctttgagagcagagtggataacaaggagagaactgtcatttctaaccttcggcctttcacattgtaccgcatcgatatccacagctgcaaccacgaggctgagaagctgggctgcagcgcctccaacttcgtctttgcaaggactatgcccgcagaaggagcagatgacattcctgggccagtgacctgggagccaaggcctgaaaactccatctttttaaagtggccggaacctgagaatcccaatggattgattctaatgtatgaaataaaatacggatcacaagttgaggatcagcgagaatgtgtgtccagacaggaatacaggaagtatggaggggccaagctaaaccggctaaacccggggaactacacagcccggattcaggccacatctctctctgggaatgggtcgtggacagatcctgtgttcttctatgtccaggccaaaacaggatatgaaaacttcatccatctgatcatcgctctgcccgtcgctgtcctgttgatcgtgggagggttggtgattatgctgtacgtcttccatagaaagagaaataacagcaggctggggaatggagtgctgtatgcctctgtgaacccggagtacttcagcgctgctgatgtgtacgttcctgatgagtgggaggtggctcgggagaagatcaccatgagccgggaacttgggcaggggtcgtttgggatggtctatgaaggagttgccaagggtgtggtgaaagatgaacctgaaaccagagtggccattaaaacagtgaacgaggccgcaagcatgcgtgagaggattgagtttctcaacgaagcttctgtgatgaaggagttcaattgtcaccatgtggtgcgattgctgggtgtggtgtcccaaggccagccaacactggtcatcatggaactgatgacacggggcgatctcaaaagttatctccggtctctgaggccagaaatggagaataatccagtcctagcacctccaagcctgagcaagatgattcagatggccggagagattgcagacggcatggcatacctcaacgccaataagttcgtccacagagaccttgctgcccggaattgcatggtagccgaagatttcacagtcaaaatcggagattttggtatgacgcgagatatctatgagacagactattaccggaaaggagggaaagggctgctgcccgtgcgctggatgtctcctgagtccctcaaggatggagtcttcaccacttactcggacgtctggtccttcggggtcgtcctctgggagatcgccacactggccgagcagccctaccagggcttgtccaacgagcaagtccttcgcttcgtcatggagggcggccttctggacaagccagacaactgtcctgacatgctgtttgaactgatgcgcatgtgctggcagtataaccccaagatgaggccttccttcctggagatcatcagcagcatcaaagaggagatggagcctggcttccgggaggtctccttctactacagcgaggagaacaagctgcccgagccggaggagctggacctggagccagagaacatggagagcgtccccctggacccctcggcctcctcgtcctccctgccactgcccgacagacactcaggacacaaggccgagaacggccccggccctggggtgctggtcctccgcgccagcttcgacgagagacagccttacgcccacatgaacgggggccgcaagaacgagcgggccttgccgctgccccagtcttcgacctgctga

SEQ ID NO: 2 - DNA, 18 bp, Artificial Sequence
  Insulin-like Growth Factor Receptor (IGF-1R) antisense oligodeoxynucleotide (AS-ODN)
tcctccggagccagactt